(19) United States
(12) Patent Application Publication
(10) Pub. No.: US 2026/0306708 A1
(43) Pub. Date: Oct. 8, 2026

(54) SYNTHETIC BIOPOLYMER SOLUTIONS WITH INCREASED LOWER CRITICAL SOLUTION TEMPERATURES

(71) Applicant: DSM IP Assets B.V., 6221 BE Maastricht (NL)

(72) Inventors: Inge Jeannette MINTEN, Geleen (NL); Jens Christoph THIES, Echt (NL)

(21) Appl. No.: 19/481,308

(22) PCT Filed: May 23, 2024
(86) PCT No.: PCT/EP2024/062266

§ 371 Date: Nov. 4, 2025

Related U.S. Application Data
(60) Provisional application No. 63/500,035, filed on May 4, 2023.

(51) Int. Cl. A61L 27/22; A61L 27/52

(52) U.S. Cl. CPC A61L 27/227; A61L 27/52; A61L 2400/06

(57) ABSTRACT

Synthetic biopolymers are disclosed, which can be engineered in terms of both their amino acid sequences and post-synthesis treatments to which they are subjected, to achieve desired gelation characteristics, particularly upon or after being exposed to physiological temperature (e.g., 37° C.), including their use in an aqueous solution including a critical solution temperature agent that increases a baseline lower critical solution temperature of synthetic biopolymer solution, for example to raise it above physiological temperature for application and, upon diffusion of the agent after application, the lower critical solution temperature drops and the synthetic biopolymer gels in the physiological environment. Examples of synthetic biopolymers are synthetic elastin-like polypeptides (ELPs) having polypeptide sequences with functional oligopeptide blocks that may include one or more hydrophobic blocks, one or more aggregation-enhancing blocks, and one or more β-sheet formation-inducing blocks.

This application contains a sequence listing that is hereby incorporated by reference.

### FIELD OF THE DISCLOSURE

Aspects of the disclosure relate to synthetic biopolymer solution compositions comprising a synthetic biopolymer and an aqueous solution that includes a critical solution temperature agent that increases the lower critical solution temperature of the synthetic biopolymer solution, for example to a temperature that is above physiological temperature, and when the agent is lost, removed, or diffused out of the synthetic biopolymer solution there is a drop in the synthetic biopolymer solution's lower critical solution temperature, which can change the properties of the biopolymer in the surrounding environment, e.g. induce gelation in the environment as the lower critical solution temperature drops below the temperature of the environment. The biopolymer may be an elastin-like polypeptide (ELP), and such compositions, which can solidify/gel after targeted administration/deposition, may be used for applications such as filler deposition, embolic, or tissue repair, such as bone void filler compositions.

### DESCRIPTION OF THE ART

Liquid embolics, which are injected as a liquid into the body but then solidify when they enter the vasculature, are used to treat a variety of diseases such as aneurysms, meningiomas, and subdural hematomas. Embolics are commonly made from polymer materials dissolved in a non-aqueous polar solvent material. The amount and strength of the solvent material, however, can complicate biological application, e.g. through deformation of the application catheter or through induced biological response such as vascospasms.

### SUMMARY

This Summary provides an introduction to some general concepts relating to this disclosure in a simplified form, where the general concepts are further described below in the Detailed Description. This Summary is not intended to identify key features or essential features of the disclosure.

In some aspects, this disclosure relates to, inter alia, synthetic biopolymer solutions, which can include a synthetic biopolymer, water, and a critical solution temperature agent. In some examples, the synthetic biopolymer solution is an aqueous solution. In certain embodiments, the critical solution temperature agent increases a baseline lower critical solution temperature of the synthetic biopolymer solution to an administration lower critical solution temperature that is above physiological temperature, and when the critical solution temperature agent diffuses out of the synthetic biopolymer solution upon exposure of the synthetic biopolymer solution to a physiological environment, the lower critical solution temperature of the synthetic biopolymer solution is lowered to an exposure lower critical solution temperature that is at or below physiological temperature. In some examples, the synthetic biopolymer is engineered to undergo gelation and physical cross-linking resulting from β-sheet formation among molecules of the synthetic biopolymer following exposure of the synthetic biopolymer solution to a physiological environment and the lowering of the lower critical solution temperature of the synthetic biopolymer solution to the exposure lower critical solution temperature.

In some examples of the synthetic biopolymer solution, the critical solution temperature agent includes or consists of dimethyl sulfoxide. In certain examples of the synthetic biopolymer solution, the critical solution temperature agent includes one or more sulfoxide moieties. In some examples, the critical solution temperature agent includes one or more hydrogen bonding moieties. In various embodiments, the critical solution temperature agent includes at least one moiety that is a sulfoxide group, an amine group, an amide group, a carbonyl group, or an alcohol group, or a combination thereof.

In certain examples, the critical solution temperature agent is present in an amount of about 0.5% to about 10%, by weight, of the synthetic biopolymer solution. In some embodiments, the critical solution temperature agent is present in an amount of about 1% to about 4%, by weight, of the synthetic biopolymer solution. In various examples, the administration lower critical solution temperature is at least about 2° C. more than the exposure lower critical solution temperature. In certain embodiments, the administration lower critical solution temperature is at least about 4° C. more than the exposure lower critical solution temperature.

In some embodiments, the administration lower critical solution temperature of the biopolymer solution is about 40° C. or higher, or is between about 41° C. and about 43° C. In some examples, the critical solution temperature agent is present in an amount that is insufficient to cause any vasospasms during an administration of the synthetic biopolymer solution. In certain embodiments, the critical solution temperature agent is present in an amount that is insufficient to swell, soften, or dissolve any latex, silicon, plastic, or rubber material used to store or administer the synthetic biopolymer solution.

In various examples of the synthetic biopolymer solution, the synthetic biopolymer is an elastin-like polypeptide. In certain embodiments, the elastin-like polypeptide includes a polypeptide sequence comprising: (a) one or more hydrophobic blocks of VPGXG (SEQ ID NO:1), wherein X represents any amino acid other than proline; (b) one or more aggregation-enhancing blocks of IPAVG (SEQ ID NO:2), and (c) one or more β-sheet formation-inducing blocks of GAGAGS (SEQ ID NO:3), GAGAGY (SEQ ID NO:4), GAGYGA (SEQ ID NO:5), or GAGAGA (SEQ ID NO:6). In some examples, X represents V or I. In certain embodiments, the polypeptide sequence further includes (d) one or more biomineralizing blocks of VTKHLNQISQSY (SEQ ID NO:7).

In some embodiments, a synthetic biopolymer solution including a synthetic biopolymer, water, and a critical solution temperature agent is disclosed, the critical solution temperature agent including dimethyl sulfoxide, and where the critical solution temperature agent is present in an amount of 0.5% to 10%, by weight, of the synthetic biopolymer solution. In these embodiments, the critical solution temperature agent increases a baseline lower critical solution temperature of the synthetic biopolymer solution to an administration lower critical solution temperature that is above physiological temperature, and when the critical solution temperature agent diffuses out of the synthetic biopolymer solution upon exposure of the synthetic biopolymer solution to a physiological environment, this lowers the lower critical solution temperature of the synthetic biopolymer solution to an exposure lower critical solution temperature that is at or below physiological temperature, where the administration lower critical solution temperature is above 37° C. and the exposure lower critical solution temperature is below 37° C. Then, the synthetic biopolymer is engineered to undergo gelation following exposure of the synthetic biopolymer solution to a physiological environment and the lowering of the lower critical solution temperature of the synthetic biopolymer solution to the exposure lower critical solution temperature. In certain embodiments, the administration lower critical solution temperature is at least about 2° C. less than the exposure lower critical solution temperature.

In some aspects of the disclosure, methods of preparing a synthetic biopolymer solution are disclosed. In some examples, a preparation method includes preparing a synthetic biopolymer engineered to undergo gelation following exposure of the synthetic biopolymer solution to a physiological environment by washing a biopolymer with an organic liquid, where the organic liquid includes an alcohol, a hydrocarbon, an ether, a carboxylic acid, an ester, or a ketone. Then, the method includes combining the synthetic biopolymer, water and an amount of a critical solution temperature agent that is effective to increase a baseline lower critical solution temperature of the synthetic biopolymer solution to an administration lower critical solution temperature that is above physiological temperature, and wherein the amount of the critical solution temperature agent is 0.5% to 10%, by weight, of the synthetic biopolymer solution.

In some aspects of the disclosure, methods of administering a synthetic biopolymer solution are disclosed. The biopolymer solution may be an aqueous solution. In some examples, the method includes administering a synthetic biopolymer solution into a physiological environment, the synthetic biopolymer solution including a synthetic biopolymer, water and a critical solution temperature agent, where the critical solution temperature agent increases a baseline lower critical solution temperature of the synthetic biopolymer solution to an administration lower critical solution temperature that is above physiological temperature, and where the critical solution temperature agent diffuses out of the synthetic biopolymer solution upon exposure of the synthetic biopolymer solution to a physiological environment, lowering the lower critical solution temperature of the synthetic biopolymer solution to an exposure lower critical solution temperature that is at or below physiological temperature. Then, the synthetic biopolymer is engineered to undergo gelation and physical cross-linking resulting from β-sheet formation among molecules of the synthetic biopolymer following exposure of the synthetic biopolymer solution to a physiological environment and the lowering of the lower critical solution temperature of the synthetic biopolymer solution to the exposure lower critical solution temperature.

In certain examples of these methods, the synthetic biopolymer solution is administered to provide a filler or a liquid embolic. In some embodiments, the synthetic biopolymer solution is administered to provide a bone filler, a dermal filler, or urinary incontinence filler.

Aspects of the disclosure are associated with the preparation of synthetic biopolymers, such as those produced by bacterial fermentation and expression in cell culture, having suitable properties for use in synthetic biopolymer solutions or other compositions that may be used as administrable or implantable compositions. Such compositions may be more particularly designed for use in implantation in a human or animal body to act as a filler or repair tissue, for example as bone void filler compositions, or for use as a liquid embolic.

These and other aspects, embodiments, and advantages relating to the present disclosure are apparent from the following Detailed Description.

### BRIEF DESCRIPTION OF THE DRAWINGS

A more complete understanding of exemplary embodiments of the invention and the advantages thereof may be acquired by referring to the following description in consideration of the accompanying figures, which serve to illustrate various features and certain principles involved.

FIG. 1 is a flow diagram of steps for preparing ELP compositions, according to particular methods as described herein.

FIG. 2 provides graphs of Ultraviolet-visible (Uv-Vis) spectroscopy absorbance and temperature data for example synthetic biopolymer solutions, and a derivate graph based on the same.

FIG. 3 provides graphs of Uv-Vis absorbance and temperature data for example synthetic biopolymer solutions.

FIG. 4 provides graphs of Uv-Vis absorbance and temperature data for example synthetic biopolymer solutions.

FIG. 5 provides graphs of LCST data based on variations in various solution properties.

FIG. 6 shows the effects of water vapor annealing of samples of synthetic ELPs.

FIG. 7 provides Fourier-transform infrared spectroscopy (FTIR) scans of aqueous solutions of synthetic ELPs, following post-synthesis treatments of freeze-drying alone or in combination with either water vapor annealing or washing with ethanol.

FIG. 8 provides graphs of changes in optical density, resulting from turbidity developed over time for aqueous solutions of synthetic ELPs at varying concentrations, introduced to a photospectrometer preheated at 37° C.

FIG. 9 shows the effects of gelation of samples of synthetic ELPs, following water vapor annealing.

FIG. 10 provides graphs of rheological properties of aqueous solutions of synthetic ELPs, before and after water vapor annealing as a post-synthesis treatment, as such properties are developed over 3 temperature cycles of (i) heating from 4° C. to 37° C. at a heating rate of 1° C. per minute and holding at 37° C. for 30 minutes, followed by (ii) cooling from 37° C. to 4° C. at a cooling rate of 1° C. per minute and holding at 4° C. for 30 minutes.

FIG. 11 provides graphs of rheological properties of aqueous solutions of synthetic ELPs, following various post-synthesis treatments of freeze-drying at different severities, including mild freeze-drying alone or in combination with water vapor annealing, and harsh freeze-drying, as such properties are developed upon heating from 4° C. to 37° C. at a heating rate of 1° C. per minute and holding at 37° C. for 4 hours.

FIG. 12 shows the microstructure of freeze-dried samples of synthetic ELPs.

FIG. 13 provides SDS PAGE analyses of synthetic ELPs, showing that post-synthesis treatments of freeze-drying alone, or in combination with either water vapor annealing or washing with ethanol, have little or no effect on purity or molecular weight of the ELP.

FIG. 14 shows the physical appearance of synthetic ELPs, following post-synthesis treatments of freeze-drying alone, or in combination with either water vapor annealing or washing with ethanol.

FIG. 15 provides dynamic light scattering (DLS) analyses of aqueous solutions of synthetic ELPs, following post-synthesis treatments of freeze-drying alone, or in combination with either water vapor annealing or washing with ethanol, showing the effect of these post-synthesis treatments on particle size, in comparison with denatured ELPs.

FIG. 16 provides graphs of rheological properties of aqueous solutions of synthetic ELPs, following various post-synthesis treatments of freeze-drying alone, or in combination with either water vapor annealing or washing with ethanol, as such properties are developed upon heating from 4° C. to 37° C. at a heating rate of 1° C. per minute and holding at 37° C. for 4 hours.

### DETAILED DESCRIPTION

Elastin like proteins can be designed to be responsive to external stimuli such as for example temperature. ELPs can undergo aggregation above a certain temperature known as the lower critical solution temperature (LCST), also sometimes referred to as lower consolute temperature. Lower critical solution temperature is the temperature where the components of a mixture are generally miscible below the temperature but are not miscible above the temperature, e.g. as one or more materials begin to or completely undergo phase separation, for example due to solidification/gelation. Below the LCST, ELPs are generally soluble in aqueous media (although some amount of gelation can occur at temperatures near the LCST) but once above the LCST they are no longer soluble and are rather a stable gel. This can lead to materials which precipitate, flocculate or by design form stable hydrogel phases.

This thermally responsive behavior can be used to create materials for application where a liquid undergoes a gelation or solidification upon injecting into the human body and warming up above the LCST. Examples could include elastin like protein formulations for injectable applications. Example applications may include dermal fillers, injectable fillers for urinary incontinence and liquid embolics. In some examples, the solutions could be used for targeted delivery and/or deposition of additional materials, such as medicaments, antibiotics, pharmaceuticals, and/or chemotherapy agents.

Particularly in terms of liquid embolics, aqueous ELP formulations will provide numerous benefits since they do not rely on substantial amounts of solvents such as DMSO to make a polymer solution capable of local precipitation and vessel occlusion. The substantial amounts of DMSO used in existing products creates problems with micro-catheter design as well as complications such as vasospasm. Aqueous ELP solutions in accordance with this disclosure avoid these issues. Furthermore, micro-catheters are very thin walled and thermal conductivity can easily mean that the ELP formulation would experience temperatures near or above the LCST, if the LCST is at or near physiologic temperature, within the microcatheter and therefore gel and block the catheter before the embolic liquid is deployed at the position where vessel occlusion is desired. Using appropriately calibrated LCST behavior of an ELP, as provided herein, avoids any premature or undesired gelation in the catheter or otherwise. Surprisingly, it has been found that use of a critical solution temperature agent such as DMSO, even in small amounts such as (but not limited to) about 0.5% to about 5% by weight in the overall aqueous biopolymer (e.g. ELP) solution can suppress the LCST behavior and inhibit gelation prior to injection. Once the formulation is injected into the human body the small amount of agent diffuses out of the injected liquid and the solution, now being mainly water based, undergoes the gelation due to the LCST behavior.

Throughout this disclosure, standard, one-letter amino acid codes are used to represent amino acid residues, including their characteristic side chains, in the indicated functional oligopeptide blocks and polypeptides in which they are incorporated. The abbreviation ELP is used for “elastin-like polypeptide” and the abbreviation ELPs is used for “elastin-like polypeptides.” A synthetic ELP is a particular example of a synthetic biopolymer, and therefore, with respect to any description herein that relates to a synthetic biopolymer, this should be understood as relating to a synthetic ELP, in preferred embodiments. Unless otherwise specified, the term “aqueous solution” in reference to an aqueous solution of a synthetic biopolymer (e.g., synthetic ELP) with about 80% or more of the solution (by weight) being made up of water. In some examples, the aqueous solution has about 85% or more water (by weight), or about 90% or more, or about 95% or more, or about 96% or more, or about 97% or more, or about 98% or more. In some examples, concentration of synthetic biopolymer in an aqueous solution has, from about 5 mg/ml to about 350 mg/ml, from about 10 mg/ml to about 300 mg/ml, from about 30 mg/ml to about 200 mg/ml, from about 50 mg/ml to about 100 mg/ml, from about 100 mg/ml to about 300 mg/ml, or from about 150 mg/ml to about 250 mg/ml, or from about 150 mg/ml to about 200 mg/ml.

In more specific embodiments, such concentration of a synthetic biopolymer (e.g., synthetic ELP) in an aqueous solution as described herein, may be at or over about 10 mg/ml, about 15 mg/ml, about 50 mg/ml, about 100 mg/ml, about 150 mg/ml, or about 200 mg/ml, or about 250 mg/ml, or more (e.g. about 150 mg/ml or more). Exemplary aqueous solutions may include, as the aqueous medium, water, distilled water, or purified water, such as Milli-Q® (MQ) water, or a salt solution, such as phosphate-buffered saline (PBS). In some examples, non-aqueous solutions with less water (or even no water) may be provided, for example solutions that use a water material or materials as a co-solvent and/or a less predominant and/or minor component of the overall solution. In some examples, synthetic biopolymer solutions may include about 20% or more water (by weight), or about 30% or more, or about 40% or more, or about 50% or more, or about 60% or more, or about 70% or more, or about 75% or more. Concentrations of a synthetic biopolymer (e.g., synthetic ELP) and critical temperature agent may be the same as discussed in embodiments that are an aqueous solution, or may be increased to account for the relatively smaller amount of water present.

Embodiments of the invention are directed to compositions including synthetic biopolymer solutions including synthetic biopolymers (e.g., synthetic polypeptides, such as synthetic ELPs) that are engineered, or configured, to undergo gelation when implanted in a human or animal body, a property that renders such synthetic biopolymers, and more specifically compositions formed from such synthetic biopolymers, suitable for embolic applications, filler use, tissue repair (e.g., for use in bone void filler compositions). In some beneficial examples, ELP or other synthetic biopolymers or proteins that can be used in the disclosure's solutions will degrade in a physiological environment. In some beneficial examples, ELP or other synthetic biopolymers or proteins that can be used in the disclosure's solutions are non-toxic. In some aspects of the disclosure, however, synthetic non-degradable polymers that are temperature sensitive such as isopropylacrylamide materials or similar materials can be used (e.g. along with a synthetic biopolymer) in a similar manner, i.e. with a critical temperature agent, to provide differences in LCST and assist in application.

In some aspects of the disclosure, synthetic biopolymer solutions are described. In some embodiments, the synthetic biopolymer solution includes a synthetic biopolymer, water and a critical solution temperature agent. In some example, the synthetic biopolymer solution is an aqueous solution. The critical solution temperature agent may consist of, include, or be based on a single material, or a combination of materials as described in more detail below. In certain embodiments, the critical solution temperature agent increases a baseline lower critical solution temperature (LCST) of the synthetic biopolymer solution to an administration lower critical solution temperature that is higher than the baseline lower critical solution temperature (LCST) that would otherwise be obtained in the absence of the critical solution temperature agent. Thus, the baseline lower critical solution temperature may be considered to correspond to that of a reference or baseline LCST of an otherwise equivalent synthetic biopolymer solution that does not contain the critical solution temperature agent and where the difference in solutions is remedied through additional water, i.e. 1% wt. water replaces 1% wt. of the critical solution temperature agent. According to particular embodiments, the critical solution temperature agent may increase a baseline lower critical solution temperature (LCST) of the synthetic biopolymer solution (i.e. the LCST value of such reference or baseline synthetic biopolymer solution) to an administration lower critical solution temperature that is around or above physiological temperature.

Below the lower critical solution temperature, biopolymers like ELPs are soluble in aqueous media but once above the LCST they are no longer soluble, e.g. as a biopolymer can precipitate, flocculate, solidify and/or gel at higher temperatures. This can advantageously facilitate targeted application of the biopolymer into a physiological environment, such as a blood vessel, or can assist with delivery to other environments and other applications.

In some examples, the critical solution temperature agent can then diffuse out of the synthetic biopolymer solution upon exposure of the synthetic biopolymer solution to a physiological environment. For example, when the synthetic biopolymer solution is injected into a particular location in the body, and is no longer in the controlled application environment, e.g., in a catheter that only contains the synthetic biopolymer solution, the critical solution temperature agent may diffuse into the greater environment and away from the deposited biopolymer material. When this occurs, the critical temperature agent's impact on the lower critical solution temperature wanes or fades entirely, often in a relatively short time period and in some embodiments essentially instantly, lowering the lower critical solution temperature of the solution. In some examples, the loss of the critical solution temperature agent results in a drop of LCST until it reaches or is below the temperature of the physiological environment. In some examples, some water of the aqueous solution also disperses and/or diffuses out, and the LCST of the biopolymer composition in the body may not exactly match the LCST of the baseline composition (e.g. due to differences in relative biopolymer concentration), but impact of changing LCST still provides a result of solidification of the synthetic biopolymer after delivery with a critical solution temperature agent that previously prevented solidification or gelation.

Put another way, the diffusion of the critical solution temperature agent may lower the lower critical solution temperature of the synthetic biopolymer solution to an exposure lower critical solution temperature, for example a temperature that is at or below physiological temperature. Thus, gelation or solidification can rapidly occur, but only in the targeted area of the physiological environment (and not, e.g. in a location along a catheter inside a body that is different from the targeted area, like a point along the path of the delivery of the synthetic biopolymer solution to the targeted area that is subjected to physiological temperature). In some embodiments such gelation or solidification can occur essentially instantly. In some embodiments, the exposure lower critical solution temperature may be identical to the baseline LCST of a reference or baseline synthetic biopolymer solution as described above. In some embodiments, the exposure lower critical solution temperature may correspond substantially to the baseline LCST of a reference or baseline synthetic biopolymer solution as described above.

In some examples, the synthetic biopolymer is engineered to undergo gelation and physical cross-linking resulting from β-sheet formation among molecules of the synthetic biopolymer following exposure of the synthetic biopolymer solution to a physiological environment and the lowering of the lower critical solution temperature of the synthetic biopolymer solution to the exposure lower critical solution temperature.

Thus, in appropriately designed embodiments using appropriately designed synthetic biopolymer and critical temperature agent, this allows deposition of the biopolymer and subsequent gelation or solidification of the biopolymer when the LCST drops below physiological temperature in the desired spot, e.g. to act as an embolic to block blood flow to, for example, a tumor. As a representative example, addition of DMSO, e.g. about 1% DMSO, to ELP in MQ water can increase the LCST of the ELP solution to an administration lower critical solution temperature that is above 37° C., such that the ELP remains in a liquid state or liquid like state at typical physiological temperatures.

In certain examples, the ELP is dissolved in MQ water or other non-salty water in the synthetic biopolymer solutions. Critical temperature agents can increase the LCST of ELP in both salty and non-salty solutions/environments, but the presence of salt (e.g. when PBS is used to prepare the solution), according to some embodiments, may depress the increase of LCST provided by the agent, requiring more agent to achieve the desired effect. In some examples, the solutions may use one or more anions or cations, e.g. hofmeigeher anions, chlorides, phosphates, or alkalis to influence the LCST of the ELP. This can be beneficial, however, as gelation is more rapid due the use of salt, e.g. gelation from a solution with PBS can occur more quickly than when MQ water is used (which can still result in rapid gelation). Thus, with appropriate ELP and agent selection and addition amounts, LCST can be increased as needed even when using a salt material like PBS, allowing quick and easy addition, while also having rapid gelation once administered.

In other embodiments, similarly rapid gelation can occur with non-salt materials like MQ water, through appropriate selection and addition amounts of ELP/temperature agent, for example adding just enough agent to increase the LCST to slightly above 37° C. (or, in this and other embodiments, to a different target temperature if the patient/subject, such as a non-human patient/subject, has a physiological temperature above or below 37° C.). In some embodiments, through appropriate selection and addition amounts of ELP/temperature agent, the LCST is increased to a temperature above physiological temperature, such as about one degree Celsius more or about two degrees Celsius more or about three degrees Celsius more (or about 0.5, or about 1.5° C., or about 2.5° C., or about 3.5° C. above 37° C. or other appropriate physiological temperature), so that no premature gelation occurs as the solution begins to approach the LCST upon application into the physiological environment, as such premature solidification or gelation can greatly frustrate or even prevent desired application.

In some embodiments, the biopolymer (e.g. ELP) is treated with an organic liquid and this can also impact LCST. For a specific example, treating an ELP with ethanol prior to use can further enhance the increased LCST temperature from the presence of a critical temperature agent, e.g. a ELP with about 2% DMSO in MQ water has an increased LCST above 37° C., but when the ELP was treated with ethanol washing first, the LCST was over 42° C. Treatment with ethanol or other appropriate organic liquids can also make the resulting gel formed more stable. In some examples, the ELP is an endotoxin rich ELP, an ethanol treated ELP, or both.

In some examples, the critical solution temperature agent comprises one or more hydrogen bonding moieties, and/or one or more moieties that disrupt hydrogen bonding between other chemical groups in the vicinity of the agent. In certain embodiments, the critical solution temperature agent comprises at least one moiety that is a sulfoxide group, an amine group, an amide group, a carbonyl group, or an alcohol group, or a combination thereof. In certain examples, the critical solution temperature agent comprises or consists of dimethyl sulfoxide (DMSO). In other examples, the agent comprises or consists of carbonyl diamide or dimethyl ketone. In some embodiments, the critical solution temperature agent comprises or consists of a compound with Lewis base functionality.

In certain embodiments, the critical solution temperature agent is present in an amount of about 0.5% to about 10%, by weight, of the synthetic biopolymer solution. In some examples, the critical solution temperature agent is present in an amount of about 1% to about 6%, by weight, of the synthetic biopolymer solution, or about 1% to about 5%, or about 1% to about 4%, or about 2% to about 4%, or about 2% to about 8%, or about 2% to about 20%. In certain embodiments, the critical solution temperature agent is present in an amount of about 50% or less, by weight, of the synthetic biopolymer solution, or about 40% or less, or about 30% or less, or about 25% or less, or about 20% or less. In various examples, the critical solution temperature agent is present in an amount of about 15% or less, by weight, of the synthetic biopolymer solution, or about 12% or less, or about 10% or less, or about 8% or less, or about 6% or less, or about 5% or less, or about 4% or less.

In some examples of the disclosure, the synthetic biopolymer solution has an administration lower critical solution temperature is at least about 2° C. more than the exposure lower critical solution temperature. For a specific example, a synthetic biopolymer solution may have an ELP and an amount of DMSO present such that the administration lower critical solution temperature is about 38° C., which is above a typical physiological temperature, and when the DMSO diffuses out the ELP material has an exposure lower critical solution temperature of about 36° C., which is below a typical physiological temperature, meaning the ELP will solidify in the physiological environment after administration. In certain embodiments, the administration lower critical solution temperature is at least about 4° C. more than the exposure lower critical solution temperature, at least about 5° C. more than the exposure lower critical solution temperature, at least about 8° C. more than the exposure lower critical solution temperature, at least about 3° C. more than the exposure lower critical solution temperature, at least about 1.5° C. more than the exposure lower critical solution temperature, at least about 1° C. more than the exposure lower critical solution temperature, at least about 0.75° C. more than the exposure lower critical solution temperature, or at least about 0.5° C. more than the exposure lower critical solution temperature.

In various examples, the administration lower critical solution temperature is at least about 1 to about 5° C. more than the exposure lower critical solution temperature, at least about 1 to about 3° C. more than the exposure lower critical solution temperature, at least about 2 to about 8° C. more than the exposure lower critical solution temperature, at least about 1 to about 10° C. more than the exposure lower critical solution temperature, at least about 2 to about 6° C. more than the exposure lower critical solution temperature, at least about 3 to about 10° C. more than the exposure lower critical solution temperature, at least about 3 to about 8° C. more than the exposure lower critical solution temperature, at least about 3 to about 6° C. more than the exposure lower critical solution temperature, or at least about 4 to about 6° C. more than the exposure lower critical solution temperature.

In some embodiments, the administration lower critical solution temperature is about 40° C. or higher, about 39° C. or higher, about 38.5° C. or higher, about 38° C. or higher, about 37.5° C. or higher, about 37° C. or higher, about 41° C. or higher, about 42° C. or higher, or about 43° C. or higher. In some examples, the administration lower critical solution temperature is between about 41° C. to about 43° C., or between about 40° C. to about 44° C., or between about 38° C. to about 44° C., or between about 38° C. to about 42° C., or between about 38° C. to about 40° C., or between about 38° C. to about 45° C.

In certain embodiments, an appropriate amount of the critical solution temperature agent is present such that the biopolymer solution will have a lower critical solution temperature that is increased, compared to an equivalent composition with an equivalent ELP (e.g., a reference or baseline solution or composition as described above) that does not contain the critical solution temperature agent, for example an increased LCST to any of the above discussed values. For an example, an ELP may have an amount of critical solution temperature agent added until the LCST is about 37° C. or more. For a more specific example, an ELP that has a LCST of about 36° C. as present in particular aqueous solution (e.g. in MQ water at a concentration of about 200 mg/ml) may have an amount of critical solution temperature agent added until the LCST is about 42° C. In some examples, the critical solution temperature agent diffuses out of the synthetic biopolymer solution upon exposure of the synthetic biopolymer solution to a physiological environment, lowering the lower critical solution temperature of the synthetic biopolymer solution to an exposure lower critical solution temperature that is at or below physiological temperature. In some examples, the critical solution temperature agent diffuses out of the synthetic biopolymer solution upon exposure of the synthetic biopolymer solution to a physiological environment, lowering the lower critical solution temperature of the synthetic biopolymer solution to an exposure lower critical solution temperature, where the administration lower critical solution temperature is above about 37° C. and the exposure lower critical solution temperature is below about 37° C.

In some examples of the synthetic biopolymer solution the synthetic biopolymer is an elastin-like polypeptide, including any of the elastin-like polypeptide examples provided herein and any elastin-like polypeptide with features described herein. While embodiments of the elastin-like polypeptide are described in more detail below, for an example, the elastin-like polypeptide may include a polypeptide sequence comprising (a) one or more hydrophobic blocks of VPGXG (SEQ ID NO:1), wherein X represents any amino acid other than proline, and/or (b) one or more aggregation-enhancing blocks of IPAVG (SEQ ID NO:2), and/or (c) one or more β-sheet formation-inducing blocks of GAGAGS (SEQ ID NO:3), GAGAGY (SEQ ID NO:4), GAGYGA (SEQ ID NO:5), or GAGAGA (SEQ ID NO:6). In some embodiments, the ELP may comprise sequences (a), (b), and (c).

In some examples of the disclosure, a synthetic biopolymer solution is provided that includes a synthetic biopolymer, water and a critical solution temperature agent comprising dimethyl sulfoxide, and where the critical solution temperature agent is present in an amount of about 0.5% to about 10%, by weight, of the synthetic biopolymer solution, and where the critical solution temperature agent increases a baseline lower critical solution temperature of the synthetic biopolymer solution to an administration lower critical solution temperature that is above physiological temperature. In certain of these examples, the critical solution temperature agent diffuses out of the synthetic biopolymer solution upon exposure of the synthetic biopolymer solution to a physiological environment, lowering the lower critical solution temperature of the synthetic biopolymer solution to an exposure lower critical solution temperature that is at or below physiological temperature. In some examples, the administration lower critical solution temperature is above about 37° C. and the exposure lower critical solution temperature is below about 37° C.

In certain examples the synthetic biopolymer is engineered to undergo gelation following exposure of the synthetic biopolymer solution to a physiological environment and the lowering of the lower critical solution temperature of the synthetic biopolymer solution to the exposure lower critical solution temperature. For example, the administration lower critical solution temperature may be at least around 2° C. more than the exposure lower critical solution temperature, which results in gelation. In some of these examples, the synthetic biopolymer solution is an aqueous solution.

In certain examples of the disclosure, the critical solution temperature agent is present in an amount that is insufficient to cause any vasospasms during an administration of the synthetic biopolymer solution, due to the beneficially small amount of critical solution temperature agent needed for appropriately designed systems in accordance with this disclosure. For example, DMSO can cause vasospasms upon application in blood vessels when sufficiently high amounts are present, an undesired side effect that lengthens and complicates medical procedures. But, for example, when smaller amounts of DMSO are present, such as about 4% or less by weight, no vasospasms will occur while beneficially and simultaneously facilitating application by increasing the LCST of the biopolymer material.

In other examples, the critical solution temperature agent is present in an amount that is insufficient to swell, soften, or dissolve any material in contact with the solution, for example latex, silicon, plastic, or rubber material used to store or administer the synthetic biopolymer solution. To use DMSO as the example again, larger amounts of DMSO can cause failures for the above reasons in many medical instruments such as catheters, and have required industry developments for, e.g. catheter design, that can greatly limit the materials that can be used, increasing cost and difficulty in medical device product design. The present compositions beneficially minimize or avoid such issues, allowing more design freedom with material choices and making catheter design for, e.g. embolics easier.

As a specific example embodiment, a composition of this disclosure may include about 1-10 wt % ELP, about 2-8 wt % DMSO, with the remaining balance water, such as PBS or MQ water, or a combination thereof. For a more specific example embodiments, a composition of this disclosure may include about 150-200 mg/mL ethanol treated ELP (such as ELP011) in MQ water with about 2-5 wt. % DMSO added.

In accordance with other aspects of the disclosures, methods are disclosed. For example, methods of preparing a synthetic biopolymer solution, e.g. an aqueous synthetic biopolymer solution, are disclosed, including methods that include preparing a synthetic biopolymer engineered to undergo gelation following exposure of the synthetic biopolymer solution to a physiological environment. The preparation may include washing a biopolymer (like an ELP) with an organic liquid, wherein the organic liquid comprises an alcohol, a hydrocarbon, an ether, a carboxylic acid, an ester, or a ketone, for example ethanol. In some examples, treating the ELP with ethanol or another organic liquid can provide a more stable gel than what would form with an equivalent ELP that is not ethanol treated, for example by inducing formation of β-sheets.

The methods may also include combining the synthetic biopolymer, water and an amount of a critical solution temperature agent (in any order) that is effective to increase a lower critical solution temperature of the synthetic biopolymer solution to an administration lower critical solution temperature that is above physiological temperature. In some examples of the method, the amount of the critical solution temperature agent is about 0.5% to about 10%, by weight, of the synthetic biopolymer solution.

For other example methods of the disclosure, the methods may relate to use of the compositions described herein. For example, a method may include administering a synthetic biopolymer solution (e.g. an aqueous synthetic biopolymer solution) into a physiological environment (e.g. a blood vessel in a human body), the synthetic biopolymer solution comprising a synthetic biopolymer, water, and a critical solution temperature agent, where the critical solution temperature agent increases a lower critical solution temperature of the synthetic biopolymer solution to an administration lower critical solution temperature that is above physiological temperature. As in other examples, in these methods the critical solution temperature agent may diffuse out of the synthetic biopolymer solution upon exposure of the synthetic biopolymer solution to a physiological environment, lowering the lower critical solution temperature of the synthetic biopolymer solution to an exposure lower critical solution temperature that is at or below physiological temperature, and where the synthetic biopolymer is engineered to undergo gelation and physical cross-linking resulting from β-sheet formation among molecules of the synthetic biopolymer following exposure of the synthetic biopolymer solution to a physiological environment and the lowering of the lower critical solution temperature of the synthetic biopolymer solution to the exposure lower critical solution temperature.

In some examples of these methods, the synthetic biopolymer solution is administered to provide an injectable filler or a liquid embolic. In certain examples, the synthetic biopolymer solution is administered to provide a bone filler, a dermal filler, or urinary incontinence filler. As a skilled artisan would appreciate, these compositions and systems allow delivery of biopolymers like ELPs for many purposes and in many locations in the body where gelled material or collagen-like material or elastin-like material would provide benefits such as increased structural integrity, blockage, or other purposes. For another representative example, the synthetic biopolymer solutions may include a contrast agent (that e.g. comprises one or more materials that provide contrast for imaging) for use in imaging as a radiopacity composition, e.g. to identify/diagnose fractures such as hairline fractures or microfractures. What's more, injecting the compositions that use low salt or no salt water (like MQ water) into an environment containing more salt that the MQ water, such as a blood vessel or other physiological location, can enhance the gelation by further driving down the LCST of the biopolymer through the new presence of salt combined with the diffusion of the critical temperature agent.

A given synthetic biopolymer that may be used in the present compositions, e.g. aqueous solutions, may be engineered to undergo gelation, following heating of a solution of said synthetic biopolymer at sub-ambient (e.g., 4° C.) or ambient temperature to physiological temperature, and undergoes physical cross-linking resulting from β-sheet formation among molecules of the synthetic biopolymer. In some examples, the synthetic biopolymer is engineered by induction of at least a portion of said β-sheet formation.

The properties of a synthetic biopolymer, such as a synthetic elastin-like polypeptide (ELP), can depend on both (i) its primary structure, according to its amino acid sequence, as well as (ii) other structures, such as its secondary structure, which can be influenced by post-synthesis treatment. For example, a synthetic ELP can be engineered or configured to achieve desired gelation characteristics, by virtue of property (1), the functional oligopeptide blocks in its polypeptide sequence, and/or property (2), post-synthesis treatment that influences secondary structure to “predispose” the synthetic ELP to physical cross-linking. In the case of property (2), such treatment can include (i) freeze-drying (lyophilization), (ii) water vapor annealing, (iii) washing with an organic liquid, and/or (iv) thermal exposure, any of which, or any combinations of which, can induce physical cross-linking such as by β-sheet formation among polypeptide molecules, thereby templating or facilitating a subsequent gelling of a composition prepared from the synthetic ELP, when such composition is implanted. Without being bound by theory, it is believed that property (1) can be engineered to principally influence gelation temperature, whereas a combination of property (1) and property (2) can be engineered to more beneficially influence gelation kinetics (e.g., such that gelation sufficiency or strength occurs over a practical time scale, which may be less than 2 hours, less than 1 hour, less than 30 minutes, or less than 15 minutes). In some examples, the synthetic biopolymer is engineered by washing with the organic liquid, wherein the organic liquid includes an alcohol (e.g. one or more of methanol, ethanol, propanol, and butanol), a hydrocarbon, an ether, a carboxylic acid, an ester, or a ketone.

A synthetic ELP can be engineered by virtue of the “design” of its primary structure, or amino acid sequence, particularly in terms of utilizing functional oligopeptide blocks in this sequence. A synthetic biopolymer may alternatively, but preferably in combination, be engineered by its post-synthesis treatment, which may comprise one or more particular steps performed after its recovery from a cell culture (e.g., after primary recovery), and/or after purification. Example synthetic biopolymers for use in this disclosure are discussed below, and any of these may be used in synthetic biopolymer solutions, including the embodiments discussed above, for example in a solution (e.g. aqueous solution) including a critical temperature agent.

Representative post-synthesis treatments of a synthetic biopolymer may alter, adjust, or tailor its gelation characteristics, such as by influencing, and preferably reducing, the time over which a composition formed from such synthetic biopolymer gels with sufficient structural rigidity as desired for an implant. For example, an aqueous solution of a synthetic biopolymer that has not been subjected to a given post-synthesis treatment and that is used as a control composition, may require a longer period to achieve a given degree of gelation (defined by rheological properties) upon heating according to a given protocol, compared to an aqueous solution of the same synthetic biopolymer, at the same concentration, which has been subjected to the post-synthesis treatment, upon heating according to the same protocol. Therefore, the use of a post-synthesis treatment may advantageously impart properties, and particularly gelation characteristics, that are desired or even necessary for an implant, which characteristics might otherwise be absent without such treatment.

Without being bound by theory, it is believed that physical cross-linking (i.e., without the need for an added cross-linking agent) can occur among molecular chains of synthetic biopolymers described herein according to various mechanisms, which include entanglement of polypeptide strands (e.g., which may be considered a one-dimensional phenomenon) as well as β-sheet formation of polypeptide strands (e.g., which may be considered a two-dimensional phenomenon) through interconnections between strands. In the case of physical cross-linking by β-sheet formation, this may be influenced or engineered, at least in part, in a given synthetic biopolymer by the incorporation of one or more β-sheet formation-inducing blocks of GAGAGS (SEQ ID NO:3), GAGAGY (SEQ ID NO:4), GAGYGA (SEQ ID NO:5), or GAGAGA (SEQ ID NO:6), which are believed to provide these interconnections and/or otherwise influence gelation characteristics. Those skilled in the art having knowledge of the present disclosure will appreciate that the number of physical cross-links that are formed (e.g., by any mechanism) can be “tuned” by adjusting various protocols, including those used for purification and formulation, following primary recovery. With respect to formulation, specific protocols that may be adjusted are those used for drying synthetic polypeptides after purification. Aspects of the invention are related to the discovery that any such adjustments, as part of a given post-synthesis treatment, can significantly influence the gelling behavior of a synthetic biopolymer (e.g., synthetic ELP). In fact, biopolymers having the same amino acid sequence can nonetheless exhibit substantially different gelation characteristics, for example as evidenced by gelling behavior determined experimentally at physiological temperature (e.g., over a range of not gelling whatsoever to forming gels with storage moduli of significantly greater than 10 kPa), depending on the extent of β-sheet formation post-synthesis, such as during purification and formulation (e.g., drying, and typically freeze-drying). According to particular embodiments, a baseline, or possibly lowest, content of β-sheets may be achieved (induced) utilizing a relatively “harsh” freeze-drying step as described herein. The obtained, freeze-dried synthetic biopolymer may then be subjected, for example, to water vapor annealing, or may then otherwise be subjected to washing with an organic liquid (e.g., ethanol), either of which additional post-synthesis treatment steps can further induce the formation of β-sheets from β-sheet formation-inducing blocks as described above (e.g., GAGAGS (SEQ ID NO:3)) in the polypeptide sequence of the synthetic biopolymer. The recognition that post-synthesis treatments can influence physical cross-linking, at least partly resulting from β-sheet formation, provides novel methods for monitoring and/or tuning mechanical properties of synthetic biopolymers to achieve desired outcomes for practical applications.

Representative post-synthesis treatments may therefore affect the secondary structure of the synthetic biopolymer and may, more particularly, cause at least some β-sheet formation among molecules of the synthetic biopolymer, for example due to the presence in these molecules of β-sheet formation-inducing block(s) (“silk” blocks) as noted above. This, in turn, can initiate, or template, physical cross-linking among these molecules (e.g., following implantation), such as by proceeding through a process of assembly of formed β-sheets, optionally in combination with entanglement of individual polypeptide strands, thereby forming physically cross-linked networks of supramolecular fibers. Advantageously, a post-synthesis treatment can be used to engineer a synthetic biopolymer, by induction of at least a portion of the β-sheet formation that ultimately accompanies gelation and that can beneficially influence the structure and strength of that gelation. Such induction can therefore effectively facilitate obtaining these desired, subsequent gelation characteristics of a synthetic biopolymer or composition comprising this synthetic biopolymer, upon exposure to elevated temperature, for example by heating of a solution of the synthetic biopolymer at sub-ambient temperature to physiological temperature. Although the post-synthesis treatment itself generally does not result in gelation, it is possible that induction of β-sheet formation by such treatment may result in physical cross-linking and/or other changes that precede and/or initiate physical cross-linking, including some filament assembly (entanglement) as described herein. A post-synthesis treatment may result in differences in properties of compositions comprising a given synthetic biopolymer, such as an increase in viscosity of an aqueous solution following such treatment, relative to that prior to such treatment. In some embodiments, β-sheet formation due to a post-synthesis treatment can be detected or confirmed using Fourier-transform infrared spectroscopy (FTIR) to scan compositions, and preferably aqueous solutions, with and without (e.g., following and prior to) such treatment. Physical cross-linking, at least partly resulting from β-sheet formation, may be induced by a given post-synthesis treatment, which in turn may comprise one or more specific post-synthesis treatment steps as described herein. This physical cross-linking, in addition to, or alternatively to, increased viscosity of an aqueous solution as noted above, may result in increased particle size in such solution. This may be detected based on a measured average particle size of a composition comprising a synthetic biopolymer (e.g., synthetic ELP) as described herein. According to other embodiments, therefore, β-sheet formation due to a post-synthesis treatment can be detected or confirmed using a suitable analytical method for measuring average particle size, such as dynamic light scattering (DLS), to analyze compositions, and preferably aqueous solutions, with and without (e.g., following and prior to) a given post-synthesis treatment. To the extent a post-synthesis treatment, comprising one or more post-synthesis treatment steps as described herein, influences interactions among molecules, its effects may be more accurately characterized as applying to a composition comprising the synthetic biopolymer, as opposed to a molecule of the synthetic biopolymer itself. In this respect, such characterization may therefore differ from that of the amino acid sequence, which is specific to the molecule.

According to particular embodiments, a synthetic biopolymer may be engineered (e.g., according to adaptations of its primary and/or secondary structure) to undergo gelation, following heating of a solution of the synthetic biopolymer at sub-ambient temperature (e.g., 4° C.) or ambient temperature (20° C.) to physiological temperature (e.g., 37° C.). Such gelation characteristics, or other gelation characteristics, may serve as a proxy for evaluating the performance of such synthetic biopolymer for its practical use in a composition designed for implantation. In this regard, to the extent that gelation characteristics may, in general, be influenced by physical cross-linking following β-sheet formation among molecules of the synthetic biopolymer, any of the gelation characteristics as described with respect to specific protocols defined herein (e.g., rheological properties obtained upon, or after, heating of a composition or subjecting a composition to one or more temperature cycles) may be used as a basis for characterization of a given synthetic biopolymer, regardless of the particular mechanism whereby such gelation characteristics are achieved.

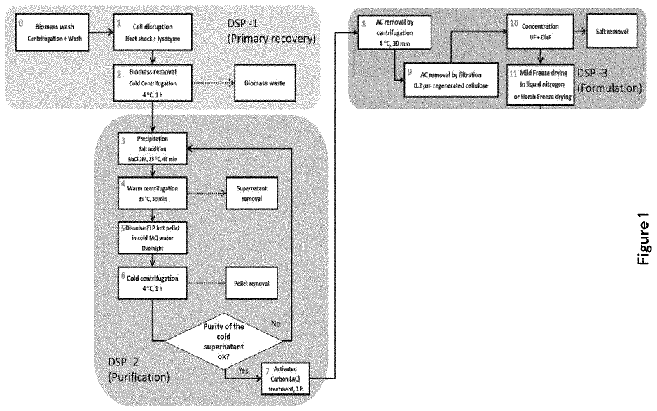

Particular examples of post-synthesis treatments, which can be used to engineer a given synthetic biopolymer by imparting characteristics as described herein, can include steps of (i) freeze-drying, (ii) water vapor annealing, (iii) washing with an organic liquid, and/or (iv) thermal exposure. Combinations of such treatment steps are also possible, such as in the case of freeze-drying, followed by water vapor annealing. A post-synthesis treatment may include any one or more of such post-synthesis treatment steps to influence gelation characteristics, with the term “post-synthesis” referring to steps occurring following the preparation of an initial synthesis composition of the synthetic biopolymer, such as following its separation and recovery from a cell culture. Typically, this separation and recovery can involve steps such as centrifugation, cell rupturing, and sonication. In one embodiment, with reference to FIG. 1, post-synthesis treatment refers to treatment steps occurring following the “Primary recovery” block, and typically within the “Purification,” “Formulation,” and “Prototype preparation” blocks. Often, post-synthesis treatment may be manipulated solely within formulation of the synthetic biopolymer and subsequent processing steps, and not within purification. For example, the step of (i) may involve the use of, and/or manipulation of, freeze-drying that occurs as part of formulation. The steps (ii) and/or (iii) may involve the use of, and/or manipulation of, water vapor annealing and/or washing with an organic liquid that may occur subsequent to a drying step (e.g., freeze-drying) that occurs as part of formulation. In the same manner, the step (iv) thermal exposure may be used and/or manipulated subsequent to a drying step, as described herein. Alternatively, or in combination, the step (iv) can include the manner in which the synthetic biopolymer is thermally exposed prior to a drying (e.g., freeze-drying) step. For example, with reference to FIG. 1, thermal exposure may include the manipulation of the “Warm centrifugation” step and more particularly the specific temperature, or the specific time-temperature profile, to which the synthetic biopolymer is exposed during this step. In this manner, and more generally, the step (iv), which may be used and/or manipulated to influence gelation characteristics as described herein, may comprise one or both of (1) a pre-drying thermal exposure and (2) a post-drying thermal exposure, with the recognition according to the present disclosure that thermal exposure occurring over the entire processing of a given synthetic biopolymer (prior to its end use) can influence gelation characteristics. According to particular embodiments, a pre-drying thermal exposure may occur before freeze-drying (e.g., in liquid nitrogen) and/or a post-drying thermal exposure may occur after freeze-drying. In other particular embodiments, a pre-drying thermal exposure or a post-drying thermal exposure, for example occurring before or after freeze-drying, may itself involve drying of a composition of a synthetic biopolymer, such as in the case of drying at elevated temperature. In this regard, FIG. 1C more specifically illustrates a step 13 of drying under vacuum at 50° C., following a step 12 of washing with an organic liquid. Insofar as step 13 occurs after freeze-drying, it may be considered a post-drying thermal exposure. It can also be appreciated that steps 12 and 13 of FIG. 1C represent post-synthesis treatments steps that are namely preparation steps (occurring in the illustrated “prototype preparation” block) for further processing the post-synthesis treated composition, with these steps having the effect of inducing physical cross-linking such as by β-sheet formation as described herein.

Certain aspects of this disclosure further relate to finding that a post-synthesis treatment for the biopolymer, and specific post-synthesis treatment steps such as (i), (ii), (iii), and/or (iv) as described herein, which can affect measurable properties that are indicative of an extent of physical cross-linking, such as occurring at least partly by β-sheet formation. These properties include, for example, viscosity, infrared absorption spectrum (e.g., FTIR spectrum), average particle size and/or particle size distribution, and gelation characteristics, any of which properties may be determined as described herein (e.g., by a suitable analysis of an aqueous solution of a given synthetic biopolymer). Accordingly, in representative methods, a post-synthesis treatment may be adjusted or modified, based on a property of a composition comprising a synthetic biopolymer as described herein, wherein such property is indicative of an extent of physical cross-linking, such as occurring at least partly by β-sheet formation. For example, according to some embodiments, in the case of an indication of low physical cross-linking or β-sheet formation (such as determined by the property of gelation characteristics that are insufficient), water-vapor annealing, washing with an organic liquid, and/or a post-drying thermal exposure may be used or manipulated (e.g., to increase severity). According to other embodiments, in the case of an indication of low physical cross-linking or β-sheet formation (for example as determined by the property of gelation characteristics that are insufficient), freeze-drying and/or a pre-drying thermal exposure may be manipulated (e.g., to increase severity). According to yet other embodiments, in the case of an indication of high physical cross-linking or β-sheet formation (for example as determined by the property of gelation characteristics that are excessive), water-vapor annealing, washing with an organic liquid, and/or a post-drying thermal exposure may be eliminated or manipulated (e.g., to decrease severity). According to still other embodiments, in the case of an indication of high physical cross-linking or β-sheet formation (for example as determined by the property of gelation characteristics that are excessive), freeze-drying and/or a pre-drying thermal exposure may be manipulated (e.g., to decrease severity). With respect to such embodiments, in the case of a post-synthesis treatment step being “used,” this refers to the implementation of such step to achieve an extent of physical cross-linking or β-sheet formation, or otherwise achieve gelation characteristics, where such post-synthesis treatment step is/was not used in a baseline, comparative, or previous post-synthesis treatment. In the case of a post-synthesis treatment step being “eliminated,” this refers to the removal of such step to achieve an extent of physical cross-linking or β-sheet formation, or otherwise achieve gelation characteristics, where such post-synthesis treatment step is/was used in a baseline, comparative, or previous post-synthesis treatment. In the case of a post-synthesis treatment step being “manipulated,” this refers to a change in severity, relative to that of the same post-synthesis step that is/was used in a baseline, comparative, or previous post-synthesis treatment. Those skilled in the art having knowledge of the present disclosure will appreciate how post-synthesis treatment steps can be manipulated to increase or decrease severity (e.g., increase a time and/or temperature of thermal exposure as described herein) and thereby regulate, i.e., increase or decrease, the extent of physical cross-linking or β-sheet formation. Embodiments of the invention are therefore directed to methods for engineering synthetic biopolymers as described herein, to achieve desired gelation characteristics, for example based on rheology measurements as described herein, with such methods comprising adjusting or modifying protocols used for preparing compositions comprising these synthetic biopolymers. The adjusting or modifying can comprise the use, manipulation, or elimination of particular post-synthesis treatment steps as described herein.

It can therefore be appreciated that a post-synthesis treatment, such as comprising one or more post-synthesis treatment steps that may include (i) freeze-drying; (ii) water vapor annealing; (iii) washing with an organic liquid; and/or (iv) thermal exposure, which may more specifically comprise one or both of (1) a pre-drying thermal exposure and/or (2) a post-drying thermal exposure; may be adjusted or modified, such as in the case of these one or more post-synthesis treatment steps being used and/or manipulated as described above, based on one or more properties indicative of an extent of physical cross-linking or β-sheet formation. Such properties include gelation characteristics, for example those a given aqueous solution of a synthetic biopolymer (e.g., synthetic ELP), which may be used as a basis for a post-synthesis treatment being adjusted or modified. Examples of determinations of gelation characteristics as being either insufficient or excessive are described herein.

Other properties, in response to which (A1) water-vapor annealing, washing with an organic liquid, and/or a post-drying thermal exposure may be used or manipulated (e.g., to increase severity), and/or (A2) freeze-drying and/or a pre-drying thermal exposure may be manipulated (e.g., to increase severity), include viscosity, infrared absorption spectrum (e.g., FTIR spectrum), average particle size and/or particle size distribution. These properties may likewise be the basis for a response according to which (B1) water-vapor annealing, washing with an organic liquid, and/or a post-drying thermal exposure may be eliminated or manipulated (e.g., to decrease severity), and/or (B2) freeze-drying and/or a pre-drying thermal exposure may be manipulated (e.g., to decrease severity). For example, a viscosity of a standard solution, such as an aqueous solution of a synthetic biopolymer (e.g., synthetic ELP) at a specific concentration and temperature, being below a threshold minimum viscosity, may be the basis for a response A1 and/or A2, whereas such viscosity being above a threshold maximum viscosity may be the basis for a response B1 and/or B2. According to other embodiments, an infrared absorption spectrum (e.g., FTIR spectrum) of a standard solution having an absorbance at a given wavenumber (e.g., at about 1622 cm⁻¹) being below a threshold minimum absorbance may be the basis for a response A1 and/or A2, whereas such absorbance being above a threshold maximum absorbance may be the basis for a response B1 and/or B2. According to yet other embodiments, with respect to an average particle size or a percentage of particles above a given particle size (e.g., in the case of a bimodal or multi-modal particle size distribution), such as measured in an aqueous solution of a synthetic biopolymer (e.g., synthetic ELP), in the case of such average particle size being below a threshold minimum particle size or such percentage being below a minimum threshold percentage, this may be the basis for a response A1 and/or A2, whereas in the case of such average particle size being above a threshold maximum particle size or such percentage being above a maximum threshold percentage, this may be the basis for a response B1 and/or B2. For example, according to particular embodiments, a threshold minimum particle size may be any discreet value within the range of 10 nanometers (nm) to 100 nm; a threshold maximum particle size may be any discreet value within the range of 100 nm to 500 nm; a minimum threshold percentage may be any discreet percentage within the range of 10% to 90%, representing the percentage of particles having a particle size above any discreet value within the range of 10 nm to 100 nm; and a maximum threshold percentage may be any discreet percentage within the range of 10% to 90%, representing the percentage of particles having a particle size above any discreet value within the range of 100 nm to 500 nm. Particle size and particle size distribution may be measured, for example, using DLS. Those skilled in the art having knowledge of the present disclosure will appreciate other specific adjustments/modifications to post-synthesis treatments that may be made in response to determinations of one or more properties indicative of physical cross-linking or β-sheet formation.

In the case of freeze-drying, this post-synthesis treatment step may comprise freezing an aqueous solution of the synthetic biopolymer and drying the frozen solution under vacuum pressure to sublimate frozen water. The severity of freeze-drying can be controlled by adjusting, for example, the surface area of frozen solution that is exposed to the vacuum conditions, the surface area-to-volume ratios of frozen volumes of the aqueous solution, the concentration of the solution, and the drying temperature. According to some embodiments, freeze-drying may, alone, be sufficient for induction of β-sheet formation to a desired extent, such that water vapor annealing or other post-synthesis treatment step, according to a given post-synthesis treatment, may not be required. According to other embodiments, freeze-drying may be used in combination with water vapor annealing, washing with an organic liquid, and/or other post-synthesis treatment step, according to a given post-synthesis treatment, to achieve induction of β-sheet formation to a desired extent, for example as determined based on any one or more properties as described herein.

A relatively more severe or “harsh” freeze-drying may comprise subjecting discreet, relatively large, frozen volumes (e.g., 35 ml frozen portions in plastic tubes) to uncontrolled temperature (e.g., ambient or room temperature) drying, whereas a relatively less severe or “mild” freeze-drying may comprise subjecting discreet, relatively small, frozen volumes (e.g., 50 μl droplets) to drying over a period, at least some portion of which (e.g., the majority of the drying period) is conducted at above-ambient temperature (e.g., 30° C.). In the case of the mild freeze-drying, the surface area-to-volume ratio of the frozen volumes is significantly higher than that as described with respect to the harsh freeze-drying. This higher surface area-to-volume ratio, combined with higher drying temperatures and/or less vacuum, may result in an overall more homogeneous process. In some cases, a mild freeze-drying can avoid the formation of a dense crust at the outer periphery of the resulting, freeze-dried volumes, which is a source of non-homogeneity that can be undesirable. According to a specific embodiment, in the case of harsh freeze-drying, 50 ml plastic tubes, each containing frozen 35 ml volumes of aqueous solution of synthetic biopolymer, are placed in a freeze-dryer maintained at 0.05 millibar (mbar) absolute pressure with a condenser temperature of −80° C. and allowed to dry for 48 hours at room temperature. According to another specific embodiment, this harsh freeze-drying may be modified to mild freeze-drying, according to which frozen droplets of the aqueous solution, for example obtained by dropwise addition of the aqueous solution into liquid nitrogen and subsequent filtration, are placed in a freeze-dryer (e.g., in a tray) maintained at 0.5 millibar (mbar) absolute pressure with a condenser temperature of −90° C., and allowed to dry according to a pre-programmed procedure, such as at a shelf (surrounding) temperature of 15° C. for 10 hours and 30° C. for 24 hours. Those skilled in the art having knowledge of the present disclosure will appreciate the variables that impact freeze-drying severity and consequently the conditions (e.g., surface area-to-volume ratio, pressure, and drying temperature) that can be altered to achieve a desired degree of homogeneity and other desired characteristics of a freeze-dried composition of a given synthetic biopolymer, considering the overall economics associated with the time and conditions required for freeze-drying under varying levels of severity.

In the case of water vapor annealing, this post-synthesis treatment may comprise exposing the synthetic biopolymer (e.g., in a solid form, such as a lyophilized form after being subjected to freeze-drying as described herein) to water vapor under vacuum conditions. For example, synthetic biopolymer in the form of a “fluffy” solid or other solid form of this material may be positioned above a water reservoir, such as placed in a tray on a support structure (e.g., disk) having multiple holes and mounted above this reservoir. A vacuum desiccator, charged with a volume of water below such support structure, is an exemplary apparatus that may be used for this purpose. Under vacuum pressure, the water evaporates and increases the surrounding humidity, causing water adsorption into the synthetic biopolymer, such that its weight may increase, for example, by at least about 50%, at about least 75%, or at least about 100%, relative to an initial weight prior to the water vapor annealing. In general, water vapor annealing also includes subsequent drying, for example in ambient air, thereby reducing moisture content. According to particular embodiments, water vapor annealing may be used as a post-synthesis treatment alone, or otherwise in combination with freeze-drying (e.g., by performing water vapor annealing before or after freeze-drying, and preferably after), for induction of β-sheet formation to a desired extent (e.g., as determined by FTIR and/or other properties as described herein, which are indicative of an extent of physical cross-linking or β-sheet formation). In the case of post-synthesis treatment comprising freeze-drying followed by water vapor annealing, the latter step may serve to further induce β-sheet formation, beyond an extent induced by the former step. Whether employed alone or in combination with one or more other post-synthesis treatment steps, and without being bound by theory, it is believed that water vapor annealing induces (or further induces) β-sheet formation as a result of the water adsorption that occurs, which imparts “plasticity” to molecules of the synthetic biopolymer, allowing them to become mobile and align β-sheet forming regions.

A further example of a post-synthesis treatment, which may be used as an alternative to one or both of freeze-drying or water vapor annealing, or which otherwise may be used in combination with one or both of these, is thermal exposure, which can likewise induce β-sheet formation when used alone, or otherwise further induce β-sheet formation in combination with other steps as described herein. In one embodiment, for example, thermal exposure can substitute for water vapor annealing, as a post-synthesis treatment step performed following freeze-drying. Like water vapor annealing, thermal exposure is also believed to increase mobility among molecules of the synthetic biopolymer, thereby allowing them to align β-sheet forming regions. Thermal exposure may comprise heating an aqueous solution of a synthetic biopolymer to an elevated temperature, generally above physiological temperature, that does not cause significant degradation or denaturing of the synthetic biopolymer. In representative embodiments, thermal exposure may comprise heating an aqueous solution of a synthetic biopolymer at a concentration from about 10 mg/ml to about 300 mg/ml, and preferably from about 100 mg/ml to about 300 mg/ml (e.g., 150 mg/ml or 250 mg/ml) to a temperature in a range from about 35° C. to about 100° C., preferably from about 50° C. to about 90° C., and more preferably from about 70° C. to about 85° C. (e.g., 80° C.). The temperature within this range (or temperature at this value) may be maintained for a thermal exposure time period sufficient for induction (or further induction) of β-sheet formation to a desired extent. In representative embodiments, this thermal exposure time period is from about 1 minute to about 12 hours, from about 1 minute to about 8 hours, from about 1 minute to about 60 minutes, from about 5 minutes to about 45 minutes, or from about 10 minutes to about 30 minutes. In general, thermal exposure may benefit from subsequent drying, for example in ambient air, thereby reducing moisture content and volume of the resulting composition (e.g., post-synthesis treated composition in an aqueous solution form).

According to particular embodiments, thermal exposure comprising heating of an aqueous solution of a synthetic biopolymer, as described above, may be a post-drying thermal exposure, i.e., performed following drying (e.g., freeze-drying) that occurs during formulation. Temperatures and times for post-drying thermal exposure include those in the ranges as described above, and this post-synthesis treatment step more broadly comprises the use of a post-drying temperature of at least about 35° C., such as at least about 50° C., and a post-drying exposure time of at least about 1 minute. According to other embodiments, thermal exposure may be more particularly a pre-drying thermal exposure, i.e., performed prior to drying (e.g., freeze-drying), such as in the case of being performed during purification (e.g., during warm centrifugation) and/or during formulation. This post-synthesis treatment step broadly comprises the use of a pre-drying temperature of at least about 35° C. (e.g., from about 35° C. to about 50° C.) and a pre-drying exposure time of at least 1 about minute (e.g., within any of the ranges described above with respect to thermal exposure time periods). With respect to manipulating a pre-drying thermal exposure and/or post-drying thermal exposure as described above, such as to increase or decrease its severity based on any one or more properties indicative of an extent of physical cross-linking or β-sheet formation, directionally increasing severity can be performed in either case by increasing the time and/or temperature of exposure (e.g., within any of the thermal exposure time ranges and/or thermal exposure temperature ranges described above), whereas directionally decreasing severity can be performed in either case by decreasing the time and/or temperature of exposure (e.g., within any of the thermal exposure time ranges and/or thermal exposure temperature ranges described above). For example, a pre-drying thermal exposure step can be made more severe by increasing the temperature of warm centrifugation, as part of synthetic biopolymer purification, and/or increasing the time over which this step of purification is performed. In general, the manipulation of a pre-drying thermal exposure step, and/or the use or manipulation of a post-drying thermal exposure step, can be performed as part of a post-synthesis treatment used to affect the extent of physical cross-linking or β-sheet formation, which in turn can be determined based on various properties (e.g., gelation characteristics, viscosity, infrared absorption spectrum, particle size and/or particle size distribution) as described herein.

A further example of a post-synthesis treatment, which may be used as an alternative to freeze-drying, water vapor annealing, and/or thermal exposure, or which otherwise may be used in combination with one or more of these, is washing with an organic liquid, which can likewise induce β-sheet formation when used alone, or otherwise further induce β-sheet formation in combination with other steps as described herein. In various embodiments, for example, washing with an organic liquid can substitute for water vapor annealing, as a post-synthesis treatment step performed following freeze-drying. Otherwise, washing with an organic liquid can be used in combination with water vapor annealing. Like water vapor annealing, washing with an organic liquid is also believed to result in dehydration of the synthetic biopolymer, as a possible mechanism for inducing β-sheet formation. Therefore, for example, a representative post-synthesis treatment may comprise drying (e.g., freeze-drying), optionally further in combination with water vapor annealing or washing with an organic liquid, or otherwise optionally further in combination with both of these post-synthesis treatment steps (e.g., water vapor annealing, followed by washing with an organic liquid).

Washing with an organic liquid may comprise contacting, for example in a batchwise or continuous manner, a dried (e.g., freeze-dried) form of the synthetic biopolymer with any suitable organic liquid that does not adversely react with the synthetic biopolymer. A representative organic liquid may be selected from the group consisting of an alcohol (e.g., methanol, ethanol, propanol, and butanol), a hydrocarbon (e.g., a C₄-C₈ alkane hydrocarbon), an ether (e.g., a dialkyl ether having C₁-C₄ alkyl groups), a carboxylic acid (e.g., having from 2 to 6 carbon atoms), an ester (e.g., having from 2-6 carbon atoms), and a ketone (e.g., dialkyl ketone having C₁-C₄ alkyl groups). A preferred organic liquid is ethanol. Representative temperatures and contacting times (e.g., residence times in the case of continuous contacting) include, respectively, approximately ambient temperature, such as from about 15° C. to about 35° C. and a range from about 1 minute to about 24 hours, such from about 1 hour to about 12 hours. Preferably, washing with an organic liquid also includes subsequent drying, for example in the case of air-drying for a time from about 1 hour to about 24 hours at approximately ambient or elevated temperature, such as from about 15° C. to about 80° C., or from about 15° C. to about 50° C., optionally under vacuum. Alternatively, as noted above with respect to FIG. 1C, such subsequent drying may otherwise be considered a separate post-synthesis treatment step of thermal exposure, which can be manipulated (e.g., by increasing or decreasing severity) to induce physical cross-linking, such as by β-sheet formation as described herein, to a desired extent. In either case, the ability of a given washing step to cause dehydration may be monitored and/or verified by measuring the moisture content of the synthetic biopolymer, for example before and after washing with the organic liquid, or otherwise monitoring vapors being driven off during the subsequent drying. A step of washing with an organic liquid may have the added benefit of removing impurities (e.g., by extracting organic compounds), such as anti-foaming agents, which may have been used during prior purification and/or formulation of the synthetic biopolymer.

In general, post-synthesis treatments, which may include one or more steps as described herein, can be used to engineer desired gelation characteristics as described herein with respect to specific protocols. Regardless of which particular treatment is employed, the induction of β-sheet formation to a desired extent may be confirmed by analytical methods for determining properties such as those described herein, including gelation characteristics and/or other properties indicative of an extent of physical cross-linking or β-sheet formation, including viscosity, infrared absorption spectrum (e.g., FTIR spectrum), or average particle size and/or particle size distribution. Such methods may be performed on a given synthetic biopolymer, or more precisely a sample thereof, obtained following its formulation, such as subsequent to drying (e.g., freeze-drying) and optionally other post-synthesis treatment steps as described herein. Those skilled in the art and having knowledge of the present disclosure can determine, for a given synthetic biopolymer, post-synthesis treatment steps and associated conditions (severity) as needed to attain an extent of induction of β-sheet formation, to influence gelation characteristics in a desired manner.

Additional embodiments of the invention are directed to synthetic ELPs having a polypeptide sequence comprising defined, functional oligopeptide blocks that are effective in synthetic biopolymers generally, for providing advantageous characteristics as described herein. Representative synthetic ELPs have a polypeptide sequence comprising: (a) one or more hydrophobic blocks of VPGXG (SEQ ID NO:1), wherein X represents any amino acid other than proline; (b) one or more aggregation-enhancing blocks of IPAVG (SEQ ID NO:2); and (c) one or more β-sheet formation-inducing blocks of GAGAGS (SEQ ID NO:3), GAGAGY (SEQ ID NO:4), GAGYGA (SEQ ID NO:5), or GAGAGA (SEQ ID NO:6). According to more particular embodiments, X in the one or more hydrophobic blocks of VPGXG (SEQ ID NO:1), such as in a portion of these hydrophobic blocks, or in all of the hydrophobic blocks, (i) may represent V, I, or E, or (ii) may represent V or I. For example, at least about 50%, at least about 60%, at least about 70%, or at least about 80% of the one or more hydrophobic blocks of VPGXG (SEQ ID NO:1) may be VPGVG (SEQ ID NO:8) and, optionally, the remainder of these blocks may be VPGIG (SEQ ID NO:9) or VPGEG (SEQ ID NO:10). The hydrophobic blocks VPGVG (SEQ ID NO:8) and VPGIG (SEQ ID NO:9), in particular, have been found to significantly affect hydrophobicity of the synthetic ELP and consequently the gelation temperature.

In some embodiments, the one or more hydrophobic blocks of VPGXG (SEQ ID NO:1) (e.g., one or more hydrophobic blocks of either VPGVG (SEQ ID NO:8) or VPGIG (SEQ ID NO:9)), may be from about 1 to about 150, from about 5 to about 120, from about 5 to about 100, or from about 25 to about 75, of these blocks. With respect to any of these ranges of hydrophobic blocks of VPGXG (SEQ ID NO:1), alternative values for the maximum stated value are 75, 60, 50, 40, 35, and 30. Therefore, for example, the above-recited range of about 1 to about 150, in alternative embodiments, may be from about 1 to about 75, from about 1 to about 60, from about 1 to about 50, from about 1 to about 40, from about 1 to about 35, or from about 1 to about 30. In other exemplary embodiments, representative synthetic ELPs may comprise (i) from about 1 to about 100, from about 1 to about 75, or from about 5 to about 60, hydrophobic blocks of VPGVG (SEQ ID NO:8) and/or (ii) from about 1 to about 80, from about 1 to about 50, or from about 2 to about 25, hydrophobic blocks of VPGIG (SEQ ID NO:9).

Alternatively, or in combination with such numbers of hydrophobic blocks and/or such percentages of particular hydrophobic blocks, the one or more aggregation-enhancing blocks of IPAVG (SEQ ID NO:2) in representative synthetic ELPs may be from about 1 to about 50, from about 1 to about 30, from about 5 to about 50, or from about 5 to about 20, of these blocks.

Alternatively, or in combination with such numbers of hydrophobic blocks, such percentages of particular hydrophobic blocks, and/or such numbers of aggregation-enhancing blocks, the one or more β-sheet formation-inducing blocks of GAGAGS (SEQ ID NO:3), GAGAGY (SEQ ID NO:4), GAGYGA (SEQ ID NO:5), or GAGAGA (SEQ ID NO:6) in representative synthetic ELPs may be from about 1 to about 50, from about 1 to about 30, or from about 5 to about 20, of these blocks. Preferably, at least about 75%, at least about 90%, or possibly all, of the one or more β-sheet formation-inducing blocks are GAGAGS (SEQ ID NO:3). The number and/or percentage of particular β-sheet formation-inducing blocks can be used to influence the extent of β-sheet formation among molecules of the synthetic ELP, when subjected to a given condition, such as a post-synthesis treatment as described herein. In some embodiments, β-sheet formation-inducing blocks (e.g., GAGAGS; SEQ ID NO:3) may represent from about 1 wt-% to about 50 wt-%, from about 3 wt-% to about 40 wt-%, from about 5 wt-% to about 35 wt-%, or from about 10 wt-% to about 20 wt-%, of the total weight of the synthetic ELP (i.e., the combined molecular weight of these functional oligopeptide blocks may represent these percentages of the total molecular weight of the synthetic ELP).

Alternatively, or in combination with such numbers of hydrophobic blocks, such percentages of particular hydrophobic blocks, such numbers of aggregation-enhancing blocks, such numbers of β-sheet formation-inducing blocks, and/or such percentages of particular β-sheet formation-inducing blocks, one or both ends of representative synthetic ELPs (i.e., a first end and/or a second end, meaning one or both termini of the molecule) may be formed exclusively by (i) at least a portion of (b) the one or more aggregation-enhancing blocks of IPAVG (SEQ ID NO:2), and/or (ii) at least a portion of (c) the one or more β-sheet formation-inducing blocks of GAGAGS (SEQ ID NO:3), GAGAGY (SEQ ID NO:4), GAGYGA (SEQ ID NO:5), or GAGAGA (SEQ ID NO:6). For example, the one or both ends may be formed exclusively by (i) at least a portion of (b) the one or more aggregation-enhancing blocks of IPAVG (SEQ ID NO:2), and/or (ii) at least a portion of (c) the one or more β-sheet formation-inducing blocks of GAGAGS (SEQ ID NO:3) in particular. It can be appreciated that an end formed exclusively by a combination of (i) and (ii) represents either a specific embodiment of this end being formed exclusively by (i), or a specific embodiment of this end being formed exclusively by (ii). For example, an end formed by (IPAVG, SEQ ID NO:2)ₓ(GAGAGS, SEQ ID NO:3)_{y}(VPGVG, SEQ ID NO:8)_{z}, in which x, y, and z are positive integers, is an example of an end formed exclusively by a combination of (i) and (ii), and this example is a specific embodiment an end formed exclusively by (i). In some cases, one end of a representative synthetic ELP may be formed exclusively by (i) at least a portion of the one or more aggregation-enhancing blocks of IPAVG (SEQ ID NO:2), and the opposite end may be formed exclusively by (ii) at least a portion of the one or more β-sheet formation-inducing blocks of GAGAGS (SEQ ID NO:3), GAGAGY (SEQ ID NO:4), GAGYGA (SEQ ID NO:5), or GAGAGA (SEQ ID NO:6), and preferably one or more of GAGAGS (SEQ ID NO:3).

Optionally, with respect to any synthetic ELP comprising functional oligopeptide blocks as defined above, and in particular embodiments with respect to synthetic ELPs having, as defined above, such numbers of hydrophobic blocks, such percentages of particular hydrophobic blocks, such numbers of aggregation-enhancing blocks, such numbers of β-sheet formation-inducing blocks, such percentages of particular β-sheet formation-inducing blocks, and/or such characteristics of one or both ends, such synthetic ELPs may optionally further comprise (d) one or more biomineralizing blocks of VTKHLNQISQSY (SEQ ID NO:7) and/or DDDEEKFLRRIGRFG (SEQ ID NO:13). The one or more biomineralizing blocks in representative synthetic ELPs may be from about 1 to about 25, from about 1 to about 20, from about 1 to about 15, or from about 1 to about 10, of these blocks. Advantageously, it has been found that such biomineralizing blocks can improve the properties of synthetic ELPs, in terms of their ability to form compositions that, in the physiological environment, sequester and/or retain constituent ions of bone mineral, namely phosphate and calcium ions, for applications as described herein (e.g., implantation to repair tissue damage, including bone defects). Compositions comprising synthetic ELPs having biomineralizing block(s) may advantageously further comprise mineral particles as described herein (e.g., hydroxyapatite and/or calcium phosphate, such as ß-tricalcium phosphate). The presence of these mineral particles, in combination with the functionality of biomineralizing block(s) to sequester phosphate ions from the bloodstream, which in turn attract and sequester calcium ions, is believed to facilitate bone growth through the mineralization process, according to which bioavailable calcium and phosphate lead to the precipitation of hydroxyapatite bone mineral.

With respect to any synthetic ELP comprising functional oligopeptide blocks as defined herein, and in particular embodiments with respect to synthetic ELPs having, as defined herein, such numbers of hydrophobic blocks, such percentages of particular hydrophobic blocks, such numbers of aggregation-enhancing blocks, such numbers of β-sheet formation-inducing blocks, such percentages of particular β-sheet formation-inducing blocks, and/or such numbers of biomineralizing blocks, these synthetic ELPs may have a molecular weight from about 10 kilo Daltons (kDa) to about 100 kDa, from about 15 kDa to about 60 kDa, from about 20 kDa to about 50 kDa, or from about 25 kDa to about 40 kDa and/or these synthetic ELPs may have an isoelectric pH, or isoelectric point pI value, from about 4 to about 11 or from about 5 to about 10.

With respect to any synthetic ELP comprising functional oligopeptide blocks as defined herein, and in particular embodiments with respect to synthetic ELPs having, as defined herein, such numbers of hydrophobic blocks, such percentages of particular hydrophobic blocks, such numbers of aggregation-enhancing blocks, such numbers of β-sheet formation-inducing blocks, such percentages of particular β-sheet formation-inducing blocks, and/or such numbers of biomineralizing blocks, these synthetic ELPs may consist of, or consist essentially of, functional oligopeptide blocks (a), (b), (c), and optionally (d), as defined herein. In some embodiments, functional oligopeptide blocks (a), (b), (c), and optionally (d), as defined herein, may represent at least about 80 wt-%, at least about 90 wt-%, at least about 95 wt-%, or at least about 99 wt-%, of the total weight of the synthetic ELP (i.e., the combined molecular weight of these functional oligopeptide blocks may represent these percentages of the total molecular weight of the synthetic ELP).

Particular synthetic ELPs of interest have the sequences [(IPAVG)₄[(VPGVG)₂(VPGIG)(VPGVG)₂]₄(GAGAGS)₄]₃ (SEQ ID NO:11, also referred to herein as “EPR011”) and [(IPAVG)₄[(VPGVG)₂(VPGIG)(VPGVG)₂]₂VTKHLNQISQSY[(VPGVG)₂(VPGIG)(VPGVG)₂]₂(GAGAGS)₄]₃ (SEQ ID NO:12, also referred to herein as “EPR018”). These and other synthetic ELPs having ends as defined above include those comprising, or consisting of, the sequence (IPAVG, SEQ ID NO:2)ₓ(VPGXG, SEQ ID NO:1)_{y}(GAGAGS, SEQ ID NO:3)_{z}, wherein x, y, and z independently represent positive integers, such as in the case of each of x, y, and z independently representing positive integers within ranges selected from those from about 1 to about 250, from about 1 to about 100, and from about 1 to about 50. Yet further synthetic ELPs having ends as defined above include those comprising, or consisting of, [(IPAVG, SEQ ID NO:2)ₓ(GAGAGS, SEQ ID NO:3)_{y}](VPGVG, SEQ ID NO:8)_{z}[(IPAVG, SEQ ID NO:2)ₘ(GAGAGS, SEQ ID NO:3)ₙ], wherein m, n, x, y, and z independently represent positive integers, such as in the case of each of m, n, x, y, and z independently representing positive integers within ranges selected from those from about 1 to about 250, from about 1 to about 100, and from about 1 to about 50.

As in the case of synthetic biopolymers generally, synthetic ELPs as described herein, by virtue of their functional oligonucleotide blocks and/or by the use of a post-synthesis treatment as described herein, may be engineered or configured to achieve desired gelation characteristics, which may generally include the ability of the synthetic biopolymer (e.g., synthetic ELP) to undergo gelation, following heating of a solution of the synthetic biopolymer (e.g., synthetic ELP) at a sub-ambient temperature (e.g., 4° C.) or ambient temperature (e.g., 20° C.) to physiological temperature (e.g., 37° C.). Particular gelation characteristics that a synthetic biopolymer (e.g., synthetic ELP) may be engineered to achieve, as described herein, include a desired temperature at which the onset of gelation occurs (e.g., according to adaptations of its primary structure) and/or desired gelation kinetics (e.g., according to adaptations of its secondary structure).

According to particular embodiments, the gelation (or gelation characteristics) of a given synthetic biopolymer (e.g., synthetic ELP) as described herein may be more concretely defined according to a protocol in which the solution is an aqueous solution comprising 150 mg/ml of the synthetic biopolymer (e.g., synthetic ELP), the sub-ambient temperature is 4° C., and the physiological temperature is 37° C. In this case, the gelation (obtained by the synthetic biopolymer) is defined by rheological properties of a gel form of the synthetic biopolymer (e.g., the synthetic ELP), obtained after the heating of the aqueous solution at 4° C., with such heating consisting of a heating rate of 1° C. per minute and a holding period at 37° C. of 4 hours. The rheological properties include a gel storage modulus (G′) exceeding a gel loss modulus (G″), with such moduli being measured, for example, e.g., in pascals (Pa). In preferred embodiments, G′ exceeds G″ by at least 10%, at least 25%, at least 50%, or at least 100%. According to particular embodiments described herein in which gelation characteristics that are insufficient may be the basis for adjusting a post-synthesis treatment, insufficient gelation characteristics may include G′ exceeding G″ by less than a threshold percentage, such as any discreet value within the range of 20% to 200%. According to other particular embodiments described herein in which gelation characteristics that are excessive may be the basis for adjusting a post-synthesis treatment, excessive gelation characteristics may include G′ exceeding G″ by more than a threshold percentage, such as any discreet value within the range of 50% to 500%.

According to certain embodiments, the gelation (or gelation characteristics) of a given synthetic biopolymer (e.g., synthetic ELP) as described herein may be, more particularly, irreversible gelation (e.g., rheological properties that define gelation do not substantially return to their original values when the starting, lower temperature is restored). As in the case of gelation generally, irreversible gelation of a given synthetic biopolymer (e.g., synthetic ELP) as described herein may be more concretely defined according to a protocol in which the solution is an aqueous solution comprising 150 mg/ml of the synthetic biopolymer (e.g., synthetic ELP), the sub-ambient temperature is 4° C., and the physiological temperature is 37° C. In the case of irreversible gelation (obtained by the synthetic biopolymer), this may be defined by rheological properties of a temperature-cycled synthetic biopolymer (e.g., temperature-cycled synthetic ELP), obtained after subjecting the aqueous solution to a temperature cycle consisting of (i) heating of the aqueous solution at 4° C., with this heating consisting of a heating rate of 1° C. per minute and a holding period at 37° C. of 30 minutes (e.g., to obtain a gel form of the synthetic biopolymer), followed by (ii) cooling from 37° C. to 4° C. at a cooling rate of 1° C. per minute and a holding period at 4° C. of 30 minutes. The rheological properties include a temperature-cycled storage modulus (TCG′) exceeding the initial aqueous solution storage modulus (IG′). In preferred embodiments, TCG′ exceeds IG′ by at least 10%, at least 25%, at least 50%, or at least 100%, meaning that the storage modulus, upon being subjected to this temperature cycle, does not return to its original value. Alternatively, but preferably in combination, the rheological properties may further include a temperature-cycled loss modulus (TCG″) exceeding the initial aqueous solution loss modulus (IG″), for example in the case of TCG″ exceeding IG″ by at least 10%, at least 25%, at least 50%, or at least 100%, meaning that the loss modulus, upon being subjected to this temperature cycle, does not return to its original value. According to particular embodiments described herein in which gelation characteristics that are insufficient may be the basis for adjusting a post-synthesis treatment, insufficient gelation characteristics (or more particularly insufficient irreversible gelation) may include TCG″ exceeding IG″ by less than a threshold percentage, such as any discreet value within the range of 20% to 200%. According to other particular embodiments described herein in which gelation characteristics that are excessive may be the basis for adjusting a post-synthesis treatment, excessive gelation characteristics may include TCG″ exceeding IG″ by more than a threshold percentage, such as any discreet value within the range of 50% to 500%.

According to other embodiments, irreversible gelation may be defined by successive increases in (or evolution of) G′ and/or G″ following each of a plurality (e.g., 2, 3, or 4) of temperature cycles as described herein, in which G′ and/or G″, obtained at the end of the holding period at 37° C. of 30 minutes, exceed its/their respective values obtained at an immediately-preceding temperature cycle, obtained at the end of the holding period at 37° C. of 30 minutes. For example, a second cycle storage modulus (2CG′) may exceed a first cycle storage modulus (1CG′) and/or a second cycle loss modulus (2CG″) may exceed a first cycle loss modulus (1CG″). In preferred embodiments, 2CG′ exceeds 1CG′ by at least 10%, at least 25%, at least 50%, or at least 100%, and/or 2CG″ exceeds 1CG″ by at least 10%, at least 25%, at least 50%, or at least 100%. Any of these differentials may likewise apply to the extent of a third cycle storage modulus (3CG′) exceeding a second cycle storage modulus (2CG′), a third cycle loss modulus (3CG″) exceeding a second cycle loss modulus (2CG″), a fourth cycle storage modulus (4CG′) exceeding a third cycle storage modulus (3CG′), a fourth cycle loss modulus (4CG″) exceeding a third cycle loss modulus (3CG″), etc. Threshold percentages by which a storage modulus of a given cycle may exceed that of a previous cycle, or by which a loss modulus of a given cycle may exceed that of a previous cycle, as the basis for gelation characteristics (or more particularly irreversible gelation) being insufficient or excessive may apply in an analogous manner as described above with respect to TCG″ exceeding IG″ by less than a threshold percentage (in the case of insufficient irreversible gelation) or by more than a threshold percentage (in the case of excessive irreversible gelation).

According to any of the protocols and associated rheological properties that define gelation, as described herein, the aqueous solution at 4° C. may have an initial aqueous solution storage modulus (IG′) substantially equal to (e.g., within about 10% of, or within about 5% of), or below, an initial aqueous solution loss modulus (IG″). According to any of the protocols and associated rheological properties that define gelation, as described herein, in some embodiments, the synthetic biopolymer would not have these associated rheological properties, absent post-synthesis treatment as described herein (e.g., according to embodiments in which post-synthesis treatment results in, or at least contributes to, advantageous gelation characteristics such as desirable structure and strength of the gelation). Rheological properties may be determined using apparatuses and their configurations and specifications, as well as any additional, specific conditions as described herein.

Further embodiments of the invention are directed to compositions comprising any synthetic biopolymer (e.g., synthetic ELP) as described herein. Representative compositions are suitable for injection and/or implantation in a human or animal body. The compositions may be provided or processed into a solution, such as an aqueous solution, and then delivered with a catheter, e.g. for delivery of liquid embolics, as discussed above.

In other embodiments, representative compositions are not necessarily immediately injectable, but can undergo further processing steps to provide such compositions. Generally, such compositions, according to embodiments of the invention, comprise a synthetic biopolymer (e.g., synthetic ELP) as described herein and may be in a solid form or an aqueous solution form, or a biopolymer solution including a critical temperature agent as discussed above. Any of these forms may have been subjected to post-synthesis treatment as described herein, for induction of β-sheet formation to a desired extent. For example, a solid form may have been subjected to freeze-drying (e.g., relatively “harsh” freeze-drying or relatively “mild” freeze-drying as described herein) and optionally water vapor annealing, washing with an organic liquid, or thermal exposure. An aqueous solution form may have been likewise subjected to one or more of these steps, as a post-synthesis treatment, and then further solubilized to provide such aqueous solution comprising the synthetic biopolymer, for example at a relatively high concentration (e.g., from about 100 mg/ml to about 300 mg/ml) for optional further processing as described herein, such as to provide an injectable or implantable composition. In other embodiments, an aqueous solution form may have been subjected, as a post-synthesis treatment, to thermal exposure as described herein, or a combination of freeze-drying and thermal exposure, with solubilization (e.g., to obtain a relatively high concentration as described above) occurring between these steps. Those skilled in the art and having knowledge of the present disclosure can appreciate that representative solid and aqueous solution forms may be obtained following any of a number of steps, including freeze-drying, water vapor annealing, washing with an organic liquid, and/or thermal exposure, which may be performed in any combination and any number of times with respect to a given, individual step (e.g., multiple cycles of freeze-drying with intermediate solubilization), for induction of β-sheet formation to a desired extent (e.g., as determined by FTIR or other properties indicative of an extent of physical cross-linking or (3-sheet formation, as described herein).

Yet further embodiments of the invention are directed to methods for preparing compositions as described herein, comprising one or more synthetic biopolymers (e.g., one or more synthetic ELPs). Representative methods of preparing synthetic biopolymers, including for use in the above noted compositions involving changes in lower critical solution temperatures through use of a diffusing temperature agent, comprise: (a) separating the synthetic biopolymer(s) from a cell culture (e.g., according to a primary recovery) and thereafter performing one or more purification steps (e.g., warm centrifugation) to provide an initial synthesis composition comprising the synthetic biopolymer(s). Those skilled in the art having knowledge of the present disclosure will appreciate that the synthetic biopolymer(s), and the initial synthesis composition, will not necessarily have the desired gelation characteristics at this stage. In this regard, however, the methods may further comprise: (b) inducing β-sheet formation among molecules of the synthetic biopolymer(s) (e.g., synthetic ELPs), to provide a post-synthesis treated composition in a solid form or an aqueous solution form. According to preferred embodiments, step (b) is carried out to an extent that does not result in gelation. According to other embodiments, β-sheet formation may be induced at least partially during purification, such that step (b) may be performed simultaneously with step (a). For example, pre-drying thermal exposure as described herein may be used or manipulated as a post-synthesis treatment step (e.g., in the warm centrifugation) for at least partially, or possibly completely, inducing β-sheet formation, to the extent obtained in the synthetic biopolymer, such as following formulation or even prototype preparation.

Whether or not all or a portion of inducing β-sheet formation (using a post-synthesis treatment as described herein) occurs in step (a) above, the solid form or aqueous solution form provided in step (b) may include any of the particular forms described herein, having been subjected to such post-synthesis treatment, in order to “engineer” a limited extent of β-sheet formation that may, for example, increase polymer chain length and increase the content of β-sheets acting to template the formation of further β-sheets. This may advantageously promote gelation and improve rheological/mechanical properties of a gel that is formed under given gelation conditions. In the case of an aqueous solution form being provided in step (b), this step may serve to increase its viscosity. In particular embodiments, step (b) may comprise freeze-drying, water vapor annealing, washing with an organic liquid, or a combination thereof, to provide the post-synthesis treated composition in the solid form. A subsequent step of solubilizing (e.g., the resulting freeze-dried and/or water vapor annealed and/or washed intermediate) may be used to provide the composition in the aqueous solution form. In other particular embodiments, step (b) may comprise freeze-drying, thermal exposure (e.g., pre-drying thermal exposure or post-drying thermal exposure), or a combination thereof, to provide the post-synthesis treated composition in the aqueous solution form, for example in the case of intermediate solubilization (e.g., following drying such as freeze-drying) occurring prior to post-drying thermal exposure. In general, although solubilization may be included at various points in a post-synthesis treatment for induction of β-sheet formation to a desired extent, solubilization alone typically does not contribute to this β-sheet formation. Solubilization, as needed to provide an aqueous form, either as an intermediate composition or the post-synthesis treated composition, may involve substantial solubilization as opposed to complete solubilization (e.g., complete solubilization may not be necessary in the practice of a given post-synthesis treatment).

### EXAMPLES

The following examples are set forth as representative of the present disclosure. These examples are not to be construed as limiting the scope of the disclosure as other equivalent embodiments will be apparent in view of the present disclosure and appended claims.

### Lower Critical Solution Temperature—Determination by UV-Vis Data

Ultraviolet-visible light spectroscopy was used to study behavior of certain example ELPS with example critical temperature agent applications. Specifically, in these example embodiments, ELP was dissolved in Milli-Q water or PBS and, for certain trials, varying concentrations of DSMO were used to perform UV-Vis studies on aqueous ELP solutions with varying DMSO concentrations.

For the general methodology of the UV-Vis experiments, 50 μL of ELP solutions were pipetted into a 384 well plate, with all sample wells surrounded by wells containing 50 μL MQ or another sample well, so that all wells experience same heat transfer from neighboring wells. A block of 3×3 wells was filled with 50 μL MQ. The well plate was placed in a refrigerator for −30 minutes, to prechill. The UV-Vis plate reader was preheated to 45° C. UV-Vis kinetic data was collected at a wavelength of 350 nm. Simultaneously the temperature within the wells was analyzed by placing thin thermocouple in the center well of the 3×3 block of wells filled with MQ water. Thermocouple was held in place by tape, data was collected by Picologger 6 app.

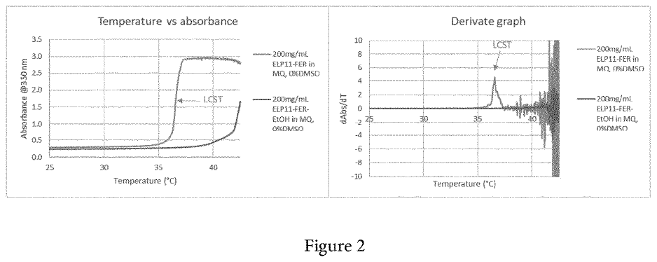

To process the data, the UV-Vis time data was matched to temperature logger time data. Data was plotted as absorbance at 350 nm vs temperature. To extract an LCST value from this data, the derivate graph was plotted. The peak value of this derivative graph corresponds to the LCST. Specifically, the LCST value was determined by plotting the derivative graph (Δ absorbance/Δ temperature on the Y-axis, temperature on the X-axis), where the LCST was taken as the temperature on the X-axis corresponding to the peak value on the Y-axis. FIG. 2 shows an illustration of temperature and absorbance data and the conversion to the derivate graph to determine the LCST.

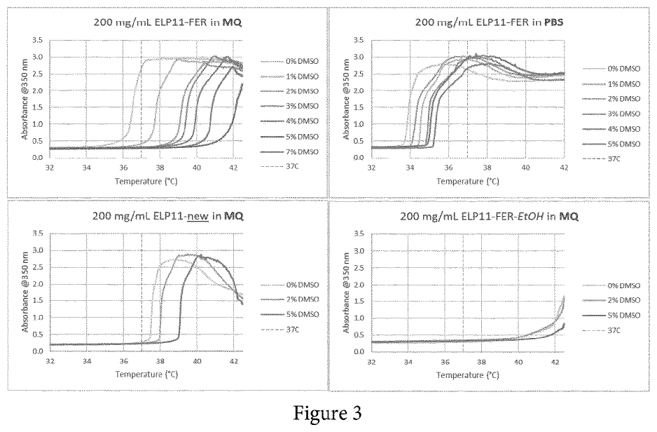

In a first set of experiments, ELP samples were dissolved in MQ water or PBS to a concentration of 250 mg/mL, and varying amounts of DMSO solution material, that would provide a final DMSO concentration as high as 7% by weight, were added to provide a final ELP concentration of 200 mg/mL in the aqueous solution containing DMSO. Samples were prepared using multiple batches of EPR011 and EPR018, which are ELP materials discussed in more detail below. For the avoidance of doubt, the graphs remove a 0 from the ELP description, e.g. ELP011 is noted as ELP11. For EPR011, “FER” in the graphs indicates an endotoxin rich ELP made by following steps 1-6 depicted in FIG. 1 with two purification cycles (steps 3-6) performed to obtain a pure product. “New” in the graphs indicates another ELP011 batch made by following steps 1-9 depicted in FIG. 1 with four purification cycles (steps 3-6) performed to obtain a pure product, the final two purification cycles being performed at a lower pH (of about 4 rather than about 6). The “new” EPR011 batch was prepared and purified after the “FER” batch. Some samples were treated with ethanol (absolute) for one hour at room temperature, followed by evaporation of the alcohol under vacuum at 50° C. overnight (this may be reflected in the graphs with “EtOH”). FIG. 3 shows the measured temperature and absorbance data and the LCST curve, with typical physiological temperature, 37° C., noted with the vertical dotted line, as this illustrates that when the LCST is above the dotted line, formulation will be substantially liquid or entirely liquid state at body temperature (as some gelation can occur prior to LCST when approaching the temperature, as shown by the increase in absorbance at very close temperatures), and further illustrates that when the LCST is below the dotted line the formulation will be in a gel state at body temperature.

As illustrated in top left chart of FIG. 3, the addition of DMSO increases the LCST of an ELP011 solution using MQ water, which is initially below typical physiological temperature such that the solution gels below typical physiological temperature without any agent added, and where the LCST increases with each increased amount of DMSO added, such that the LCST greatly exceeds 37° C., including a LCST value around 42° C. or more for the highest concentration of DMSO in these particular examples, 7%. The bottom left chart shows a similar result for another ELP011 solution using MQ water, where increased amounts of DMSO increase LCST.

The top right chart shows an ELP011 solution in PBS solution, which shows a similar trends based on addition of DMSO, but also has lower LCST values, even compared to a solution of the same ELP material using MQ water (top left). Because PBS more closely resembles physiological conditions than MQ Water, e.g. through the presence of salts, this illustrates how in certain embodiments MQ Water may be used to increase the LCST of the solution as it may be applied (e.g. in a catheter that only contains the MQ Water/ELP based solution), and then transition to the physiological environment can drop the LCST, inducing gelation or making gelation easier, with or without the use of an additional LCST agent. Thus, in some examples, choice of the aqueous material may intentionally raise or lower LCST of the ELP solution, and then optionally may be paired with an appropriate amount of a LCST agent to target a particular LCST range or value. Thus, in some embodiments, MQ water, distilled water, or other non-salt solutions may be used for the aqueous material to increase the LCST, and then the LCST agent may be added to further increase the LCST, such that the LCST is well above physiological temperature and ensures little to no premature gelation until application to the body, where exposure and diffusion will induce LCST gelation at the desired location. The bottom left chart of FIG. 3 shows the impact of ethanol treatment, which substantially raised the LCST of these solutions, and the LCST was further increased with each addition of DMSO. Thus, treatment with ethanol or other organic liquids, or other processing treatments, may be used to also control the LCST, in conjunction with or instead of use of a particular water type and/or type and amount of LCST agent.

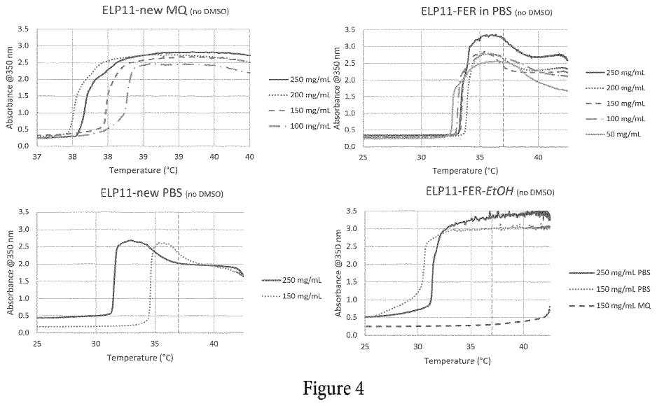

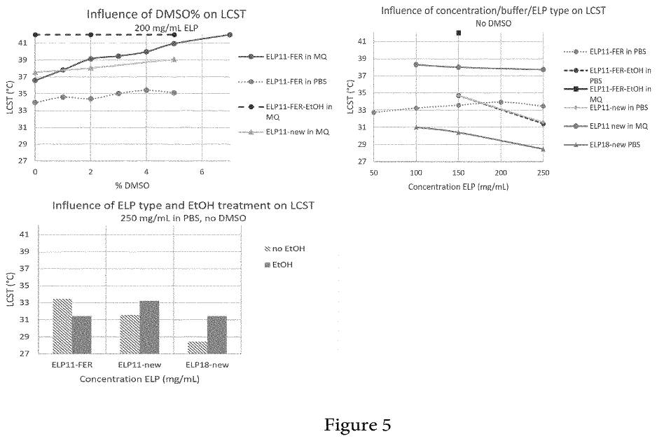

FIG. 4 shows additional example data further illustrating the potential impacts of water type on biopolymer solutions, e.g. an increase of LCST through use of MQ water compared to PBS. FIG. 4 also shows how the techniques described herein can have different impacts depending on ELP type, for example with these solutions of ELP011-FER material ethanol treatment appears to lower LCST rather than raise it. Thus, any processing technique may be used to raise of lower LCST of an ELP material, as appropriate depending on how that material responds to the treatment, although it appears more typical that MQ water use, and ethanol treatment, both increase LCST of ELP solutions. FIG. 5 shows an example data further illustrating LCST impacts based on variation in processing treatment, water type, concentration of ELP, and amount of DMSO added.

Tables 1-4 below also provides LCST values of various example solutions. Underlined values indicate LCST below 37° C., i.e. the ELP solution is thus expected to be gelled at body temperature. Italicized values indicate LCST above 37° C., i.e. the ELP solution is thus expected to be in generally or entirely in a liquid state at body temperature (and entirely liquid when no increased absorbance is measured). Values for PBS without DMSO for ELP11-new and ELP11-FER-EtOH are average values of the measured solutions at 250 and 150 mg/mL, as these solutions were not measured at 200 mg/mL.

TABLE 1
200 mg/mL

| | | | ELP11- | |
|---|---|---|---|---|
| | ELP11- | ELP11- | FER- | ELP11- |
| DMSO | FER in | FER in | EtOH | new in |
| % | MQ | PBS | in MQ | MQ |
| 0 | 36.58 | 33.94 | >42 | 37.56 |
| 1 | 37.82 | 34.59 | | |
| 2 | 39.13 | 34.39 | >42 | 38.06 |
| 3 | 39.46 | 35.01 | | |
| 4 | 39.97 | 35.40 | | |
| 5 | 40.95 | 35.09 | >42 | 39.09 |
| 7 | >42 | | | |

TABLE 2
no DMSO

| | | ELP11- | ELP11- | | | |
|---|---|---|---|---|---|---|
| ELP | ELP11- | FER- | FER- | ELP11- | ELP11 | ELP18- |
| (mg/ | FER in | EtOH | EtOH | new in | new in | new |
| mL) | PBS | in PBS | in MQ | PBS | MQ | PBS |
| 250 | 33.46 | 31.43 | | 31.57 | 37.71 | 28.45 |
| 200 | 33.94 | | | | | |
| 150 | 33.57 | 34.69 | >42 | 34.69 | 38.00 | 30.38 |
| 100 | 33.23 | | | | 38.29 | 31.00 |
| 50 | 32.72 | | | | | |

TABLE 3
250 mg/mL in PBS, no DMSO

| | ELP11- | ELP11- | ELP18- |
|---|---|---|---|
| | FER | new | new |
| no | 33.46 | 31.57 | 28.45 |
| EtOH | | | |
| EtOH | 31.43 | 33.20 | 31.43 |

TABLE 4
200 mg/mL

| | 0% | 2% | 0% |
|---|---|---|---|
| | DMSO | DMSO | DMSO |
| | in MQ | in MQ | in PBS |
| ELP11-FER | 36.58 | 39.13 | 33.94 |
| ELP11-new | 37.56 | 38.06 | 33.13 |
| ELP11- | >42 | >42 | 33.06 |
| FER-EtOH | | | |

As illustrated with the above example results, addition of DMSO can increase LCST, as can using MQ instead of PBS as a buffer. Addition of DMSO to ELP in MQ can therefore increase the LCST such that the ELP remains a solution with low yield stress at 37° C., and upon injecting in PBS (a substitute for injecting in the body), the LCST decreases to below body temperature causing the ELP to form a solid gel. Both addition of DMSO, and the use of MQ are therefore advantageous in many aspects of this disclosure. It is noted that the LCST values are slightly different between the 2 batches of ELP011 measured here, but not to an extent that it would substantially impact the described mechanism of application of biopolymer solutions based on this ELP, of these or other batches. But as these differences illustrate, in an appropriate batch or type of ELP, need addition of DMSO or other similar agent may not be needed for at least some applications, as changing the buffer from MQ to PBS might be sufficient to adjust the LCST to the degree needed. What's more, ethanol treatment of the ELPs can be beneficial in certain embodiments, as it can drives the LCST up even higher in MQ water, thereby further delaying gelation. When dissolved in PBS, ethanol treatment appears to increase LCST for the new ELPs, while for the old (ELP11-FER) batch LCST decreases. In MQ however, the LCST of the ELP11-FER batch increases with ethanol treatment. It is also noted that the shape of the UV-Vis curve changes upon ethanol treatment. Without ethanol treatment the absorbance can decrease somewhat after ELP has gone through LCST, and again around 40° C., while with ethanol treatment it can stay more stable at the elevated level. Possibly, ELP does not form a stable gel without ethanol treatment and phase separates, resulting in this profile. As an example corroboration, visual observation in one set of tests confirmed that samples treated with ethanol all visibly formed a gel. Thus, treatment with organic liquids, or other processes to induce sheet formation, can beneficially provide a stable gel. It is also noted that ELP concentration can impact LCST. For the new ELP11 and ELP18, increasing concentration appears to decrease LCST, while for the old (ELP11-FER) batch LCST slightly increases with increasing concentration. LCST of a solution may also be influenced by blending two or more synthetic biopolymers having differing LCSTs.

As examples of synthetic biopolymers for use in solutions, synthetic elastin-like polypeptides, EPR011 and EPR018 were designed and synthesized. EPR011 has the sequence [(IPAVG)₄[(VPGVG)₂(VPGIG)(VPGVG)₂]₄(GAGAGS)₄]₃ (SEQ ID NO:11). It was formed by the following blocks: VPGVG (SEQ ID NO:8), a canonical ELP block inspired in the hydrophobic repetitive and temperature-responsive block found in human tropoelastin; VPGIG (SEQ ID NO:9), variation of the VPGVG (SEQ ID NO:8) block in which the 4^{th} amino acid is substituted by isoleucine, a more hydrophobic amino acid that aims at obtaining ELPs that coacervate at physiological conditions; IPAVG (SEQ ID NO:2), a plastic-like ELP block to enhance aggregation upon temperature increase; and GAGAGS (SEQ ID NO:3), a silk-like block that enables the formation of β-sheet crystalline structures in EPR011 gels that render those gels irreversible while increasing their mechanical strength. The expected molecular weight of EPR011 was 34.9 and the isoelectric point, pI, was 5.3. EPR018 has the sequence [(IPAVG)₄[(VPGVG)₂(VPGIG)(VPGVG)₂]2VTKHLNQISQSY[(VPGVG)₂(VPGIG)(VPGV G)₂]₂(GAGAGS)₄]₃ (SEQ ID NO:12). It was formed by the blocks as described above with respect to EPR011, but included the following, additional block: VTKHLNQISQSY (SEQ ID NO:7), a biomineralizing block with the ability to template the growth of hydroxyapatite for bone regeneration purposes. The expected molecular weight of EPR018 was 39.1 and the isoelectric point, pI, was 9.7.

### Strain Construction

Synthetic genes encoding EPR011 or EPR018 were ordered from a commercial supplier. The received plasmids were transformed into E. coli according to the supplier's protocol. The transformants were plated on 2*PY agar+antibiotic. Incubation was performed at 30° C. overnight. Afterwards, clones were selected and stored in glycerol stocks (50%) at −80° C. until further use.

### Fermentation and Protein Expression

According to steps performed in primary recovery, E. coli transformants expressing EPR011 or EP018 were inoculated in 2*PY+antibiotics (100 ng/μl neomycin) and incubated at 30° C. and 250 rpm overnight. The next day, 1/100 volume of preculture in 2*PY was inoculated for the fermentative production of EPR011 or EPR018 in 500 ml shake flasks with 100 ml of Terrific Broth (TB) medium+antibiotics (100 ng/μl neomycin). Cells were incubated at 37° C. and 250 rpm for ca. 3-4 hours. Once the optical density of the culture reached 0.6-0.8, the cells were induced with L-arabinose to a final concentration of 0.02% and incubated at 27° C. overnight. Cells were harvested the day after, using 50 ml conical tubes by centrifugation at 7186 relative centrifugal force (rcf) for 20 minutes at 4° C., and the obtained pellets were stored at −20° C. until further use, to enhance cell lysis due to the freezing and subsequent thawing, and to minimize protein degradation.

### Purification and Formulation of EPR011

The frozen pellets obtained from fermentation were thawed on ice. Once the material was completely thawed, the cells were resuspended in PBS and the pH was adjusted to 4.0. The suspension was then subjected to a heatshock step at 90° C. for 30 minutes to facilitate cell lysis and ELP release, as well as protease inactivation. Thereafter, the suspension was cooled down to 25° C., the pH was adjusted to 6.0, and 0.6 g/kg lysozyme (chicken egg white) was added to enhance ELP release. The liquid was slowly cooled down overnight to 4° C. under constant stirring. The resulting suspension was then centrifugated at 7186 rcf for 1 h at 4° C. to separate the ELP (suspended in the cold supernatant) from the residual biomass. The cold supernatant was separated and NaCl was added to it, to achieve a molarity of 1 M. According to a warm centrifugation step, the aqueous solution was placed in a water bath preheated at 35° C. for 45 min. This caused the coacervation of EPR011, which was separated from the liquid by centrifugation at 30° C. for 30 min at 7186 rcf. The hot supernatant was discarded, and the pellet (containing mostly EPR011) was re-suspended in half of the starting volume of cold Milli-Q® (MQ) water and maintained at 4° C. overnight under constant stirring to facilitate the resuspension of EPR011 in cold water. The following day, a cold centrifugation at 7186 rcf for 1 h at 4° C. was performed to further purify EPR011, which remained in the cold supernatant. The material was then analyzed by UPLC or SDS PAGE (described below) to confirm the purity. It can be appreciated that this purification cycle can be repeated if the purity is not satisfactory. According to these purification steps, an aqueous solution containing dissolved EPR011, based on protein content, was obtained.

This aqueous solution was subjected to a concentrating (and if needed a diafiltration) step using a 1 kDa regenerated cellulose membrane to remove any leftover salts and other small contaminants, possibly present in the suspension. The resulting liquid product was then added in a drop-wise manner to liquid N₂ to facilitate the formation of frozen EPR011 droplets/pellets (ca. 0.5 in diameter). This facilitated the freeze-drying of EPR011 by increasing the surface area of the frozen EPR011 and thereby promoting the removal of water by lyophilization. The obtained frozen droplets were placed in a Christ freeze-dryer with a slow drying program, and a final EPR011 dry product was obtained after 2 days. The purity was thereafter checked with Total Amino Acid (TAA) analysis.

### Purification and Formulation of EPR018

The synthetic ELP, EPR018, was purified in substantially the same manner as EPR011, although it was recognized that protease inactivation from the heatshock step was beneficial in terms of preventing degradation of EPR018 due to severing of the biomineralizing block in its polypeptide sequence. In addition, the cold supernatant, which was separated from the residual biomass, was adjusted to pH 10, prior to the NaCl addition.

### Preparation Method Overview

As in the case of EPR011, EPR018 at this point was recovered as a freeze-dried composition, following the same mild freeze-drying as described above for EPR011, which included freezing droplets in liquid N₂. The mild freeze-drying in each case was therefore part of the post-synthesis treatment. According to another procedure, this mild freeze-drying was replaced with harsh freeze-drying, and preparations using this procedure are designated “CFD” in the figures. In the case of harsh freeze-drying, 50 ml plastic tubes, each containing frozen 35 ml volumes of aqueous solution of EPR011 or EPR018 were placed in a freeze-dryer maintained at 0.05 millibar (mbar) absolute pressure with a condenser temperature of −80° C., and allowed to dry for 48 hours at room temperature. With respect to either of the freeze-drying procedures, steps involved in primary recovery, purification, and formulation, leading up to obtaining the freeze-dried compositions of EPR011 and EPR018, are labeled as steps 1-11 in the flow diagram of FIG. 1A. Steps 1 and 2 in these figures can be considered steps providing an initial synthesis composition, prior to post-synthesis treatment.

Water Vapor Annealing, Washing with Ethanol, and Thermal Exposure

The freeze-dried compositions of EPR011 and EPR018, obtained as described above, were subjected to water vapor annealing using an isotemp vacuum oven (13 mbar) overnight (ca. 18 h) at room temperature to induce the formation of β-sheets from the GAGAGS (SEQ ID NO:3) blocks in the ELP sequence. Afterward, the water-annealed material was air-dried overnight at room temperature. Like the mild freeze-drying or harsh freeze-drying used to prepare the synthetic biopolymers, the water vapor annealing was also part of (i.e., a step in) the post-synthesis treatment. As an alternative to water vapor annealing, in other procedures, washing with ethanol was used. Thermal exposure can be an alternative post-synthesis treatment step for induction of β-sheet formation to a desired extent. An alternative sequence of post-synthesis treatment steps may include freeze-drying and washing with an organic liquid (in this case ethanol). Subsequent drying at elevated temperature can be considered part of the washing with organic liquid, or otherwise can be considered a separate post-synthesis treatment step of thermal exposure, to the extent that drying itself can be manipulated to influence gelation characteristics

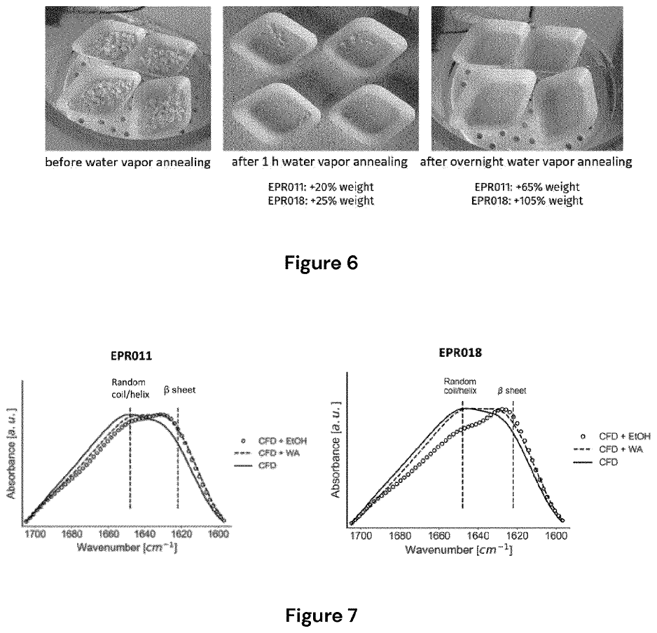

The water vapor annealing step resulted in an observable change in morphology of both EPR011 and EPR018, as shown in FIG. 6. During the water vapor annealing step, the ELPs adsorbed a large amount of water (+65 wt-% for EPR011, +105 wt-% for EPR018), which resulted in a marked change in the secondary structure of the ELPs, as shown by FTIR. Likewise, this secondary structure was also found to be influenced by washing with ethanol. In this regard, FIG. 7 depicts FTIR scans of aqueous solutions of EPR011 and EPR018, following post-synthesis treatments of (i) harsh freeze-drying alone (“CFD”), (ii) harsh freeze-drying+water vapor annealing (“CFD+WA”), and (iii) harsh freeze-drying+washing with ethanol (“CFD+EtOH”). Based on the data shown in this figure, the differing post-synthesis treatment steps could be used to achieve dramatic changes in secondary structure of the ELPs, in terms of β-sheet formation.

### Characterization and Results

Total amino acid (TAA) analysis: TAA analysis was done according using Waters Accq Tag method after chemical hydrolysis.

Ultra-High Performance Liquid Chromatography (UPLC): Detection of ELPs by UPLC was performed in a Waters HClass-Bio UPLC system (LC905) with a URP-UV 220 nm detector and using trifluoroacetic acid (TFA) as an ion paring agent. A Waters RP C4 column (1.7 μm, 50×2.1 mm) was used. The mobile phase A consisted of 100% MQ water and 0.05% TFA, while the mobile phase B consisted of 10% MQ water/90% acetonitrile plus 0.04% TFA. The system was operated at a flow rate of 0.4 ml/min and a column temperature of 20° C.

Thermogravimetric analysis (TGA): Thermogravimetric analysis (TGA) was performed on a Mettler Toledo DSC/TGA. Approximately 5 mg of sample was weighed into a pre-weighed aluminum oxide cup of 70 μl. The temperature program profile consisted of the following steps: Step 1, heating from 25 to 100° C. at a heating rate of 5° C./minute; Step 2, holding at 100° C. for 10 minutes; Step 3, heating from 100 to 200° C. at a heating rate of 5° C./minute; Step 4, holding at 200° C. for 10 minutes; Step 5, heating from 200 to 1000° C. at a heating rate of 40° C./minute; Step 6, holding at 1000° C. for 5 minutes; Step 7, cooling from 1000 to 25° C. at a cooling rate of 40° C./minute; Step 8, holding at 25° C. for 10 minutes. Moisture content was determined by the weight loss at 100° C. (step 2), and ash content was determined by the weight loss at 25° C. (step 8).

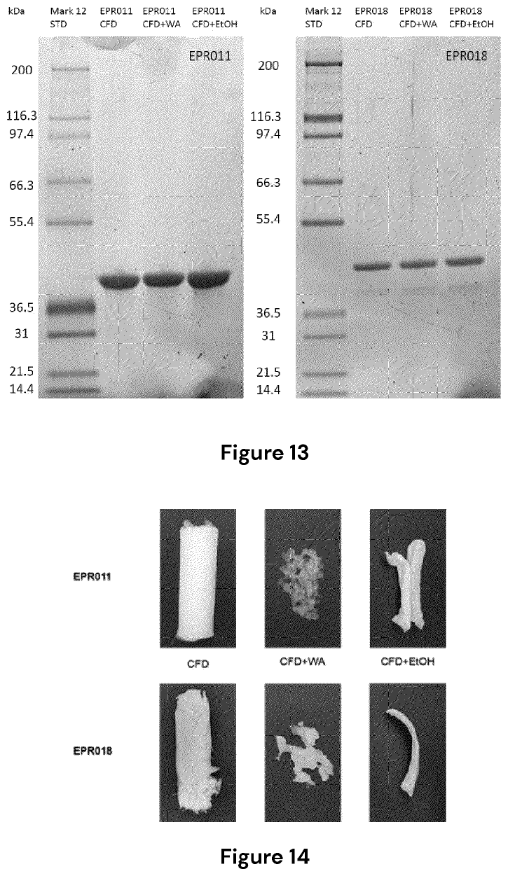

SDSPAGE: Sodium dodecyl sulphate-polyacrylamide gel electrophoresis (SDS-PAGE) was used to assess purity of synthetic biopolymers using NuPAGE 4-12% Bis-Tris gels from Invitrogen and following the protocol from the manufacturer. Each SDSPAGE sample was incubated at 70° C. for 10 minutes before analysis. Two gels were run using MOPS solution, for 50 min with 200 V, and Mark 12™ was used as the protein ladder. The resulting gel was stained with Sypro Red, using 30 ml of acetic acid (7.5 wt-%) and 6 μl of Sypro Red staining agent. FIG. 13 shows SDS-PAGE gels obtained for EPR011 and EPR018, in addition to these synthetic biopolymers following post-synthesis treatments of (i) harsh freeze-drying alone (“CFD”), (ii) harsh freeze-drying+water vapor annealing (“CFD+WA”), and (iii) harsh freeze-drying+washing with ethanol (“CFD+EtOH”), as described above. Advantageously, the different ways in which these polypeptides were processed did not materially affect their purities or molecular weights. This is despite the fact that the post-synthesis treatments (i), (ii), and (iii) resulted in differences in physical appearance of the dried materials, as shown in FIG. 14.

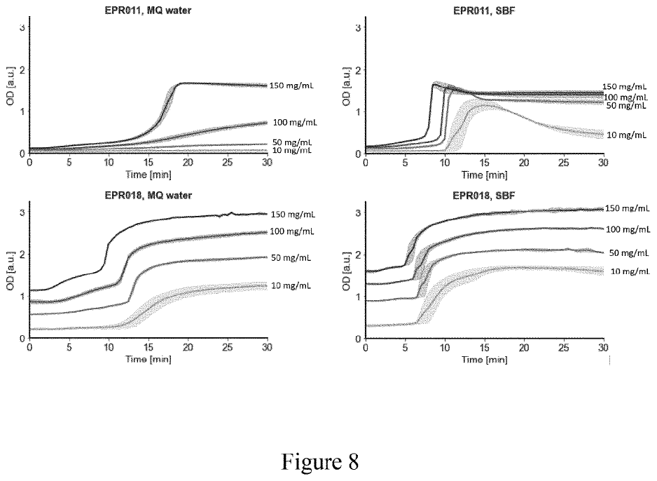

Optical density (OD): Optical density measurements were performed using aqueous ELP solutions at varying polypeptide concentrations (10, 50, 100, and 150 mg/ml) and in two different solvents: MQ water and simulated body fluid (SBF). In each case, 0.1 ml samples of aqueous solution were loaded onto 96-well plates, and the plates were then introduced to a photospectrometer preheated at 37° C. The changes in optical density, or turbidity, were recorded over time. These changes for aqueous solutions of EPR011 and EPR018 are shown in FIG. 8. Based on the graphs in this figure, the increases in turbidity occur over a shorter time period for EPR018 than for EPR011. Also, higher concentration for a given ELP directionally led to faster changes in turbidity.

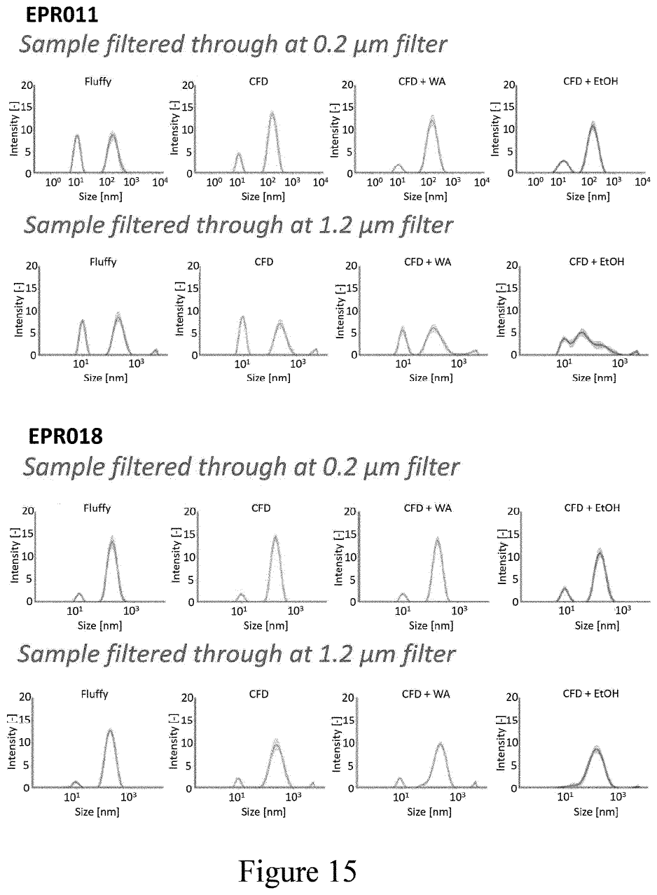

Dynamic Light Scattering (DLS): The particle size distribution of 0.5 mg/ml solutions of synthetic biopolymers was measured using a Zetasizer Nano Series dynamic light scattering (DLS) instrument (Malvern Instruments). Samples were dissolved in water and incubated at 4° C. for 1 hour. Subsequently, they were filtered using a 0.2 μm or 1.2 μm syringe filter, prior to analysis. Measurements were performed in plastic PS cuvettes (BrandTech Scientific) at 25° C. The laser power was adjusted automatically by the built-in auto-attenuation capability for each sample to an optimized range of counts. The acquisition time for each data point was 10 seconds, and 5 replicas were acquired per sample. FIG. 15 shows the DLS results obtained for 0.5 mg/ml solutions EPR011 and EPR018, in addition to these synthetic biopolymers following post-synthesis treatments of (i) harsh freeze-drying alone (“CFD”), (ii) harsh freeze-drying+water vapor annealing (“CFD+WA”), and (iii) harsh freeze-drying+washing with ethanol (“CFD+EtOH”), as described above. This figure provides the particle size distributions using the two types of filters, 0.2 μm or 1.2 μm, at 25° C.

The DLS analysis allows for the assessment of how different post-synthesis treatments in formulation and/or prototype preparation, affected the particle sizes of the diluted, 0.5 mg/ml, solutions of EPR011 and EPR018. The results indicated that CFD+WA and CFD+EtOH treatments increased the content of particles with larger diameters, providing evidence that indicates that WA or EtOH, as post-synthesis treatment steps, formed physical cross-links between polypeptide chains, thereby increasing the effective chain length of the biopolymers in solution. This facilitated the formation of a percolated network upon gelation, which was a factor in ultimately providing robust and irreversible gels. Further supporting the proposed phenomena, and illustrated in FIG. 15, is the finding that denaturing with 6M guanidine hydrochloride disrupts β-sheet formation among polypeptide strands, leading to only a single peak in solution, corresponding to the hydrodynamic diameter of a single ELP molecule.

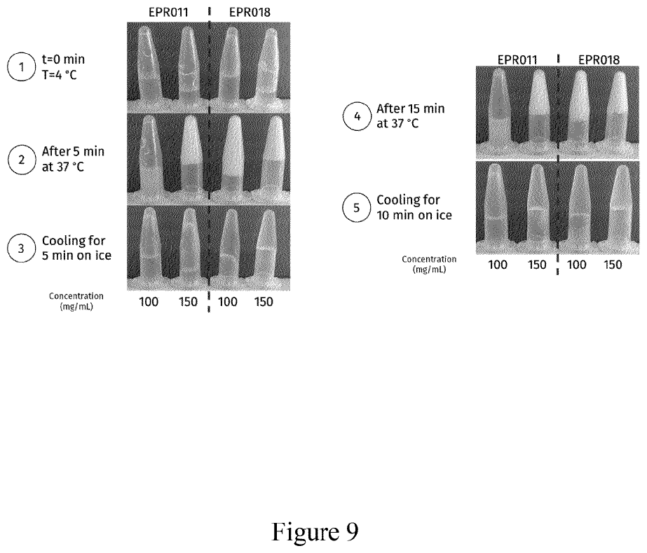

Gelation: Gelation was estimated through an inverted test tube technique. According to this procedure, aqueous solution volumes of ca. 1 ml with ELP concentrations of 100 mg/ml and 150 mg/ml were tested. The aqueous solutions were placed in Eppendorf tubes of 1.5 ml in volume, and subjected to 37° C. for 15 minutes. For these tests, samples of EPR011 and EPR018 after water vapor annealing were used, because these ELPs, when subjected to the relatively “mild” freeze-drying alone and without water vapor annealing, did not lead to irreversible gelation in the time scales used for these experiments. The results in FIG. 9 show the time evolution of the irreversibility of these ELPs after incubation. The water vapor annealed EPR018 formed irreversible gels after 5 min at 37° C. Those gels were able to maintain their shape, even after cooling on ice. The water vapor annealed EPR011 needed slightly more time, namely 15 min at 37° C., to form irreversible gels.

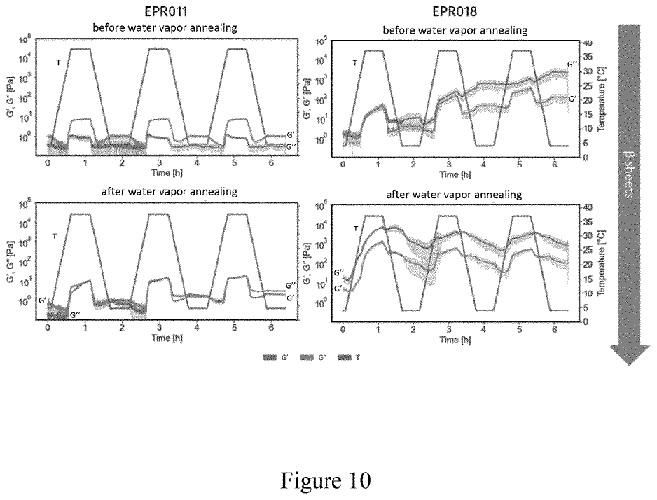

Shear rheology: Rheological characterization was performed to assess the ability of solutions of synthetic biopolymers to form hydrogels, and to characterize their viscoelastic properties. More specifically, the linear viscoelastic moduli of aqueous solutions containing EPR011 or EPR018, both before and after water vapor annealing that followed freeze-drying procedures (mild or harsh) as described above, were measured by small amplitude oscillatory shear rheology on a stress-controlled rheometer (Anton Paar MCR 301), equipped with a cone-plate geometry having a diameter of 50 mm, and cone angle of 1°. The experiments were performed at temperatures between 4 and 37° C., set by a Peltier system. Aqueous ELP solutions of 590 μl having 150 mg/ml (15 wt-%) polypeptide concentration were loaded onto the bottom plate with a pipette and at a temperature of 4° C., and these solutions were allowed to thermally equilibrate for 5-10 minutes. Low viscosity mineral oil (Sigma Aldrich) was applied to air-sample interfaces around the measuring geometry to prevent water evaporation. Rheological properties (storage modulus, loss modulus, and phase angle) were determined by applying an oscillatory shear strain at an oscillation frequency of 1 Hz, and a small strain amplitude of 0.3%. The temperature was increased from 4 to 37° C. at a rate of 1° C./minute, and held at 37° C. for 30 minutes, before bringing the temperature back again to 4° C. at a rate of 1° C./min, and maintaining 4° C. for an additional 30 minutes. This temperature cycle was repeated 3 times in total. The data in FIG. 10 show the evolution of the storage modulus (G′) and loss modulus (G″) of gels made from the aqueous solutions of EPR011 and EPR018, before and after water vapor annealing and following the same, mild freeze-drying procedure, over the temperature cycles. Based on this data, the water vapor annealing step impacted the ability of ELPs to form irreversible gels. In the case of EPR011, aqueous solutions of this polypeptide could not form a structured gel without water vapor annealing, i.e., only following this post-synthesis treatment step was it able to do so. Also, the gels became stiffer with every temperature cycle. For EPR018, the water vapor annealing step did not seem to provide any significant advantage, in terms of mechanical properties of the irreversible gel. However, this post-synthesis treatment step did clearly accelerate the formation of an irreversible gel, which attained much higher mechanical strength following the first temperature cycle after water vapor annealing.

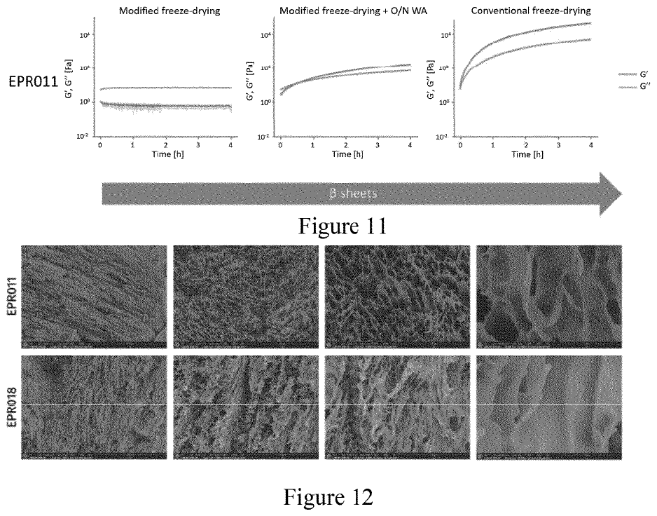

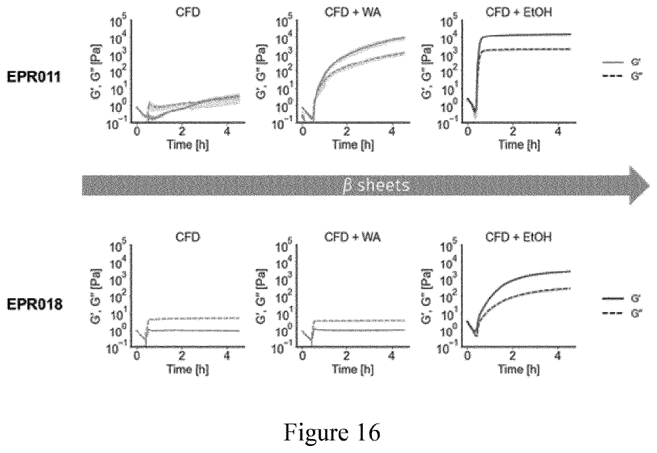

In other experiments, a “time sweep” temperature profile was used, according to which the temperature was increased from 4 to 37° C. at a rate of 1° C./minute, and held at 37° C. for 4 hours. The data in FIG. 11 show the changes over time for the storage modulus (G′) and loss modulus (G″) obtained for aqueous solutions of EPR011, following post-synthesis treatments of (i) mild freeze-drying alone (“Modified freeze-drying”), (ii) mild freeze-drying+water vapor annealing (“Modified freeze-drying+O/N WA), and (iii) harsh freeze-drying alone (“Conventional freeze-drying”). The same rheological properties over time were also measured for aqueous solutions of EPR011 and EPR018, following post-synthesis treatments of (i) harsh freeze-drying alone (“CFD”), (ii) harsh freeze-drying+water vapor annealing (“CFD+WA”), and (iii) harsh freeze-drying+washing with ethanol (“CFD+EtOH”), as described above, and the results are shown in FIG. 16. Based on the data from FIGS. 7 and 13, the formation of a stable gel, which can be indicated by G′ exceeding G″ upon heating, could be achieved using various post-synthesis treatments as described herein, although satisfactory gel formation does not necessarily result in all cases (e.g., in certain cases of using a mild freeze-drying procedure alone). As is evidenced by the results shown in FIGS. 3, 12, and 13, the effect of differing post-synthesis treatments, involving different steps following freeze-drying, can be determined based on a number of properties. Differing extents of β-sheet formation or content, are shown by FTIR, and these are further apparent from larger diameters obtained from cross-linking of synthetic biopolymers, shown by DLS. The FTIR and DLS results support a finding that physical cross-linking, at least partly resulting from β-sheet formation, influences the ability of synthetic biopolymers described herein to form gels, in addition to the mechanical properties of those gels. Importantly, the results demonstrate that CFD+WA and CFD+EtOH can be used in a post-synthesis treatment to form a desired degree of β-sheets in a given synthetic biopolymer, and even to tailor such biopolymer to achieve required gelation characteristics.

Scanning Electron Microscopy (SEM): ELP samples (in the form of films or hydrogels) were coated with gold using a vacuum coater, before imaging with an SEM.

Raman spectroscopy: The ELP samples were measured using a Renishaw inVia Raman microscope equipped with a 523 nm laser at 100% power, using an objective 50×LWD and an acquisition speed of 5×ls.

Fourier Transform Infrared Spectroscopy (FTIR): Dry samples of synthetic biopolymers (in the form of films or hydrogels) were analyzed by FTIR to assess their secondary structure. Infrared spectra were measured in a Bruker Vertex 70 Attenuated Total Reflectance FTIR device equipped with a Harrick split pea accessory. For each measurement, 64 scans with a resolution of 2 cm⁻¹ were coded in the range of 650 to 4000 cm⁻¹. The secondary structure of the polypeptides is related to the C═O stretching vibration and can be determined by performing peak deconvolution over the amide I region (1595-1705 cm⁻¹). This was performed using the lmfit package for curve fitting from Python. The peak positions were allowed to shift 4 cm⁻¹ to obtain a reconstituted curve as close as possible to the original spectra. The amide I region from all spectra was normalized to its highest value, to facilitate the comparison between different samples. The Levenberg-Marquardt least-squares method was used for fitting, and a Gaussian model was selected for the band shape.

Secondary structure characterization of ELPs: Freeze-dried EPR011 and EPR018 samples were evaluated via FTIR spectroscopy to evaluate the secondary structure of these synthetic polypeptides before and after water vapor annealing. This was done to understand the effect of water vapor annealing on the conformation of the starting ELP material, prior to dissolution in water. From this data, it could be observed that water vapor annealing can add to the content of β-sheets formed after freeze-drying whether performed using a mild freeze-drying step or a harsh freeze-drying step.

Microstructure characterization of ELP hydrogels: Freeze-dried samples of certain ELP aqueous solutions that showed a gelling behavior in the shear rheology experiments were analyzed by SEM to investigate their microstructure. Initially, samples of these aqueous solutions, 80 μl in volume, were incubated in microcentrifuge tubes at 37° C. for 1 hour. The resulting hydrogels were submerged in liquid N₂ for 1 min, followed by lyophilization. The freeze-dried samples were then cryo-fractured and imaged via SEM. As shown in FIG. 12, these samples of EPR011 and EPR018 had an open porous structure, with no clear differences in porosity. These results indicated that the inclusion of a biomineralizing domain in EPR018 did not cause its microstructure to vary appreciably, compared to that of EPR011.

Overall, aspects of the disclosure relate to compositions comprising synthetic biopolymers (e.g., synthetic ELPs) having desirable properties or solutions of such synthetic biopolymers, for example by being suitable for use in implantable compositions to act as fillers or embolics or to repair tissue, such as in bone void filler compositions. In some embodiments, the synthetic biopolymers can replace collagen used in conventional compositions. Those skilled in the art having knowledge of the present disclosure will recognize that various changes can be made to the disclosed synthetic biopolymer solutions, synthetic biopolymers, compositions, methods of preparation, and methods of use, in attaining these and other advantages, without departing from the scope of the present disclosure. As such, it should be understood that the features of the disclosure are susceptible to modifications and/or substitutions, without departing from this scope.

These materials, compositions and process descriptions are merely examples. In certain embodiments, the compositions include additional combinations and/or substitutions of some or all of the components described above. Moreover, additional and alternative suitable variations, forms and components for the compositions will be recognized by those skilled in the art given the benefit of this disclosure. Finally, any of the features discussed in the example embodiments of the processes may be features of embodiments of the compositions (or components thereof), and vice versa.

The specific embodiments illustrated and described herein are not limiting of the disclosure as set forth in the appended claims.

1. A synthetic biopolymer solution comprising:
a synthetic biopolymer, water, and a critical solution temperature agent;
wherein the synthetic biopolymer solution is an aqueous solution;
wherein the critical solution temperature agent increases a baseline lower critical solution temperature of the synthetic biopolymer solution to an administration lower critical solution temperature that is above physiological temperature;
wherein upon exposure of the synthetic biopolymer solution to a physiological environment, the critical solution temperature agent diffuses out of the synthetic biopolymer solution and thereby lowers the lower critical solution temperature of the synthetic biopolymer solution to an exposure lower critical solution temperature that is at or below physiological temperature; and
wherein said synthetic biopolymer is engineered to undergo gelation and physical cross-linking resulting from β-sheet formation among molecules of the synthetic biopolymer following exposure of the synthetic biopolymer solution to a physiological environment and the lowering of the lower critical solution temperature of the synthetic biopolymer solution to the exposure lower critical solution temperature.

2. The synthetic biopolymer solution of claim 1, wherein the critical solution temperature agent comprises or consists of dimethyl sulfoxide.

3. The synthetic biopolymer solution of claim 1, where the critical solution temperature agent is present in an amount of 0.5% to 10%, by weight, of the synthetic biopolymer solution.

4. The synthetic biopolymer solution of claim 2, where the critical solution temperature agent is present in an amount of 1% to 4%, by weight, of the synthetic biopolymer solution.

5. The synthetic biopolymer solution of claim 1, wherein the administration lower critical solution temperature is at least 2° C. more than the exposure lower critical solution temperature.

6. The synthetic biopolymer solution of claim 5, wherein the administration lower critical solution temperature is at least 4° C. more than the exposure lower critical solution temperature.

7. The synthetic biopolymer solution of claim 1, wherein the critical solution temperature agent comprises one or more hydrogen bonding moieties.

8. The synthetic biopolymer solution of claim 7, wherein the critical solution temperature agent comprises at least one moiety that is a sulfoxide group, an amine group, an amide group, a carbonyl group, or an alcohol group, or a combination thereof.

9. The synthetic biopolymer solution of claim 1, wherein the administration lower critical solution temperature is 40° C. or higher.

10. The synthetic biopolymer solution of claim 1, wherein the administration lower critical solution temperature is between 41° C. and 43° C.

11. The synthetic biopolymer solution of claim 1, wherein the synthetic biopolymer is an elastin-like polypeptide.

12. The synthetic biopolymer solution of claim 11, wherein the elastin-like polypeptide comprises a polypeptide sequence comprising:
(a) one or more hydrophobic blocks of VPGXG (SEQ ID NO:1), wherein X represents any amino acid other than proline;
(b) one or more aggregation-enhancing blocks of IPAVG (SEQ ID NO:2); and
(c) one or more β-sheet formation-inducing blocks of GAGAGS (SEQ ID NO: 3), GAGAGY (SEQ ID NO:4), GAGYGA (SEQ ID NO:5), or GAGAGA (SEQ ID NO:6).

13. A synthetic biopolymer solution comprising a synthetic biopolymer, water, and a critical solution temperature agent, wherein the critical solution temperature agent comprises dimethyl sulfoxide and is present in an amount of 0.5% to 10%, by weight, of the synthetic biopolymer solution;
wherein the critical solution temperature agent increases a baseline lower critical solution temperature of the synthetic biopolymer solution to an administration lower critical solution temperature that is above physiological temperature;
wherein the critical solution temperature agent diffuses out of the synthetic biopolymer solution upon exposure of the synthetic biopolymer solution to a physiological environment, lowering the lower critical solution temperature of the synthetic biopolymer solution to an exposure lower critical solution temperature that is at or below physiological temperature, wherein the administration lower critical solution temperature is above 37° C. and the exposure lower critical solution temperature is below 37° C.; and
wherein said synthetic biopolymer is engineered to undergo gelation following exposure of the synthetic biopolymer solution to a physiological environment and the lowering of the lower critical solution temperature of the synthetic biopolymer solution to the exposure lower critical solution temperature.

14. The synthetic biopolymer solution of claim 13, wherein the administration lower critical solution temperature is at least 2° C. more than the exposure lower critical solution temperature.

15. A method of preparing the synthetic biopolymer solution according to claim 13, the method comprising:
preparing a synthetic biopolymer engineered to undergo gelation following exposure of the synthetic biopolymer solution to a physiological environment by washing a biopolymer with an organic liquid, wherein the organic liquid comprises an alcohol, a hydrocarbon, an ether, a carboxylic acid, an ester, or a ketone; and
combining the synthetic biopolymer, water, and a critical solution temperature agent to provide a synthetic biopolymer solution, wherein the critical solution temperature agent is added in an amount that is effective to increase a baseline lower critical solution temperature of the synthetic biopolymer solution to an administration lower critical solution temperature that is above physiological temperature, and wherein the amount of the critical solution temperature agent is 0.5% to 10%, by weight, of the synthetic biopolymer solution.

16. A method comprising:
administering a synthetic biopolymer solution into a physiological environment, the synthetic biopolymer solution comprising a synthetic biopolymer, water, and a critical solution temperature agent;
wherein the synthetic biopolymer solution is an aqueous solution;
wherein the critical solution temperature agent increases a baseline lower critical solution temperature of the synthetic biopolymer solution to an administration lower critical solution temperature that is above physiological temperature;
wherein the critical solution temperature agent diffuses out of the synthetic biopolymer solution upon exposure of the synthetic biopolymer solution to a physiological environment, lowering the lower critical solution temperature of the synthetic biopolymer solution to an exposure lower critical solution temperature that is at or below physiological temperature; and
wherein said synthetic biopolymer is engineered to undergo gelation and physical cross-linking resulting from β-sheet formation among molecules of the synthetic biopolymer following exposure of the synthetic biopolymer solution to a physiological environment and the lowering of the lower critical solution temperature of the synthetic biopolymer solution to the exposure lower critical solution temperature.

17. The method of claim 16, wherein the synthetic biopolymer solution is administered to provide a filler or a liquid embolic.

18. The method of claim 16, wherein the synthetic biopolymer solution is administered to provide a bone filler, a dermal filler, or urinary incontinence filler.

19. The synthetic biopolymer solution of claim 1, wherein the critical solution temperature agent is present in an amount that is insufficient to cause any vasospasms during an administration of the synthetic biopolymer solution.

20. The synthetic biopolymer solution of claim 1, wherein the critical solution temperature agent is present in an amount that is insufficient to swell, soften, or dissolve any latex, silicon, plastic, or rubber material used to store or administer the synthetic biopolymer solution.


SEQUENCE LISTING (13 sequences)

SEQ ID NO: 1 - protein, 5 aa, synthetic construct
  VARIANT 4: note = hydrophobic block, Xaa = Any Amino Acid Other Than Proline
VPGXG

SEQ ID NO: 2 - protein, 5 aa, synthetic construct
  REGION 1..5: note = aggregation-enhancing block
IPAVG

SEQ ID NO: 3 - protein, 6 aa, synthetic construct
  REGION 1..6: note = beta sheet formation-inducing block
GAGAGS

SEQ ID NO: 4 - protein, 6 aa, synthetic construct
  REGION 1..6: note = beta sheet formation-inducing block
GAGAGY

SEQ ID NO: 5 - protein, 6 aa, synthetic construct
  REGION 1..6: note = beta sheet formation-inducing block
GAGYGA

SEQ ID NO: 6 - protein, 6 aa, synthetic construct
  REGION 1..6: note = beta sheet formation-inducing block
GAGAGA

SEQ ID NO: 7 - protein, 12 aa, synthetic construct
  REGION 1..12: note = biomineralizing block
VTKHLNQISQSY

SEQ ID NO: 8 - protein, 5 aa, synthetic construct
  REGION 1..5: note = hydrophobic block
VPGVG

SEQ ID NO: 9 - protein, 5 aa, synthetic construct
  REGION 1..5: note = hydrophobic block
VPGIG

SEQ ID NO: 10 - protein, 5 aa, synthetic construct
  REGION 1..5: note = hydrophobic block
VPGEG

SEQ ID NO: 11 - protein, 432 aa, synthetic construct
  REGION 1..432: note = Elastin-like polypeptide EPR011
IPAVGIPAVGIPAVGIPAVGVPGVGVPGVGVPGIGVPGVGVPGVGVPGVGVPGVGVPGIGVPGVGVPGVGVPGVGVPGVGVPGIGVPGVGVPGVGVPGVGVPGVGVPGIGVPGVGVPGVGGAGAGSGAGAGSGAGAGSGAGAGSIPAVGIPAVGIPAVGIPAVGVPGVGVPGVGVPGIGVPGVGVPGVGVPGVGVPGVGVPGIGVPGVGVPGVGVPGVGVPGVGVPGIGVPGVGVPGVGVPGVGVPGVGVPGIGVPGVGVPGVGGAGAGSGAGAGSGAGAGSGAGAGSIPAVGIPAVGIPAVGIPAVGVPGVGVPGVGVPGIGVPGVGVPGVGVPGVGVPGVGVPGIGVPGVGVPGVGVPGVGVPGVGVPGIGVPGVGVPGVGVPGVGVPGVGVPGIGVPGVGVPGVGGAGAGSGAGAGSGAGAGSGAGAGS

SEQ ID NO: 12 - protein, 468 aa, synthetic construct
  REGION 1..468: note = Elastin-like polypeptide EPR018
IPAVGIPAVGIPAVGIPAVGVPGVGVPGVGVPGIGVPGVGVPGVGVPGVGVPGVGVPGIGVPGVGVPGVGVTKHLNQISQSYVPGVGVPGVGVPGIGVPGVGVPGVGVPGVGVPGVGVPGIGVPGVGVPGVGGAGAGSGAGAGSGAGAGSGAGAGSIPAVGIPAVGIPAVGIPAVGVPGVGVPGVGVPGIGVPGVGVPGVGVPGVGVPGVGVPGIGVPGVGVPGVGVTKHLNQISQSYVPGVGVPGVGVPGIGVPGVGVPGVGVPGVGVPGVGVPGIGVPGVGVPGVGGAGAGSGAGAGSGAGAGSGAGAGSIPAVGIPAVGIPAVGIPAVGVPGVGVPGVGVPGIGVPGVGVPGVGVPGVGVPGVGVPGIGVPGVGVPGVGVTKHLNQISQSYVPGVGVPGVGVPGIGVPGVGVPGVGVPGVGVPGVGVPGIGVPGVGVPGVGGAGAGSGAGAGSGAGAGSGAGAGS

SEQ ID NO: 13 - protein, 15 aa, synthetic construct
  REGION 1..15: note = biomineralizing block
DDDEEKFLRRIGRFG